SEQUENCE LISTING (6 sequences)

SEQ ID NO: 1 - DNA, 23 bp, synthetic construct
  misc_feature 1..23: note = forward primer of the first primer pair
  source 1..23: mol_type = other DNA
aakagcctgcrtacktcaycctc

SEQ ID NO: 2 - DNA, 23 bp, synthetic construct
  misc_feature 1..23: note = reverse primer of the first primer pair
  source 1..23: mol_type = other DNA
gttggagaggtgggcaggggtca

SEQ ID NO: 3 - DNA, 23 bp, synthetic construct
  misc_feature 1..23: note = forward primer of the second primer pair
  source 1..23: mol_type = other DNA
crttgcgcatgatgtgaastgac

SEQ ID NO: 4 - DNA, 25 bp, synthetic construct
  misc_feature 1..25: note = reverse primer of the second primer pair
  source 1..25: mol_type = other DNA
atgrrggsagacatgtttayttgaa

SEQ ID NO: 5 - DNA, 23 bp, synthetic construct
  misc_feature 1..23: note = forward primer of the third primer pair
  source 1..23: mol_type = other DNA
acasrgcacctcsaarccctcyt

SEQ ID NO: 6 - DNA, 23 bp, synthetic construct
  misc_feature 1..23: note = reverseprimer of the third primer pair
  source 1..23: mol_type = other DNA
acaagcagtgggtcactaaggtc

(19) United States
(12) Patent Application Publication
(10) Pub. No.: US 2026/0327696 A1
(43) Pub. Date: Oct. 22, 2026

(54) METHOD FOR GENOTYPING FULL-LENGTH SEQUENCES OF 16 KIR GENES BASED ON THIRD-GENERATION LONG-READ SEQUENCING

(71) Applicants: Shenzhen Blood Center (Shenzhen Institute of Transfusion Medicine), Shenzhen, Guangdong (CN); Shenzhen Baoan Women's and Children's Hospital, Shenzhen, Guangdong (CN)

(72) Inventors: Zhihui DENG, Shenzhen, Guangdong (CN); Jianxin ZHEN, Shenzhen, Guangdong (CN); Yunan LI, Shenzhen, Guangdong (CN)

(73) Assignees: Shenzhen Blood Center (Shenzhen Institute of Transfusion Medicine), Shenzhen, Guangdong (CN); Shenzhen Baoan Women's and Children's Hospital, Shenzhen, Guangdong (CN)

(21) Appl. No.: 19/537,123

(22) Filed: Feb. 11, 2026

(30) Foreign Application Priority Data
Apr. 9, 2025 (CN) 202510438090.0

(51) Int. Cl. C12Q 1/686

(52) U.S. Cl. CPC C12Q 1/686

(57) ABSTRACT

Proposed is a method for third-generation long-read sequencing-based typing of full-length sequences of 16 KIR genes. Three pairs of specific primers were designed for all 16 KIR genes in the KIR gene cluster, based on the structure of full-length sequences of KIR genes, the distribution of single nucleotide polymorphisms, and the polymorphisms of KIR alleles in the Chinese population, and the full-length sequences of all 16 KIR genes in humans were amplified using a long-range PCR method. The method for high-throughput third-generation long-read sequencing-based typing of KIR genes first established by the present disclosure has the characteristics of simple experimental operation for amplification and library construction, low cost, and no ambiguous results, and thus has broad application prospects in fields such as population genetics, disease association, and bone marrow transplantation.

### CROSS-REFERENCE TO RELATED APPLICATION

This patent application claims the benefit and priority of Chinese Patent Application No. 202510438090.0 filed with the China National Intellectual Property Administration on Apr. 9, 2025, the disclosure of which is incorporated by reference herein in its entirety as part of the present application.

### REFERENCE TO SEQUENCE LISTING

A computer readable XML file entitled “sequencelisting”, which was created on Jan. 26, 2026 and has a file size of about 8,738 bytes, contains the sequence listing for this application and is hereby incorporated by reference in its entirety.

### TECHNICAL FIELD

The present disclosure relates to the field of DNA sequencing-based typing, and in particular to a method for genotyping full-length sequences of 16 KIR genes based on third-generation long-read sequencing.

### BACKGROUND

Natural killer (NK) cells are an important component of the innate immune system in the human body. Killer immunoglobulin-like receptors (KIRs), expressed on the surface of NK cells and some activated T cells, interact with human leukocyte antigen (HLA) class I molecules on the surface of target cells to form KIR-HLA receptor-ligand complexes. The complexes transduce activating or inhibitory signals, jointly regulate the activity of NK cells, and play an important role in tumor immunity, maternal-fetal immunity, and defense against infections in the body.

KIR receptors can be classified into KIR2D and KIR3D based on the number of extra-cellular domains. KIRs are classified into inhibitory (L-type) and activating (S-type) types based on the length of the cytoplasmic region and the presence or absence of the immune receptor tyrosine-based inhibitory motif (ITIM).

The KIR gene cluster encoding KIR receptors, located on human chromosome 19, contains 14 functional KIR genes (2DL1, 2DL2, 2DL3, 2DL4, 2DL5, 2DS1, 2DS2, 2DS3, 2DS4, 2DS5, 3DL1, 3DL2, 3DL3 and 3DS1) and two pseudogenes (2DP1 and 3DP1)^{[1]}. In the international IPD-KIR database^{[2]} (https://www.ebi.ac.uk/ipd/kir/), the genomic full-length sequences of the 14 included functional KIR genes range from 9,901 bp to 17,009 bp, and the genomic full-length sequences of the two KIR pseudogenes 2DP1 and 3DP1 are 13,126 bp and 5,713 bp, respectively. The full-length coding sequences (CDSs) of each functional KIR gene are only 915-1,368 bp, whereas the non-coding sequences are as long as 8,773-15,641 bp (see Table 1). In-depth analysis of the fragment size of the full-length sequence of each KIR gene and design of specific primers to amplify amplicons with similar target fragment sizes are crucial for ensuring the success of multiplex amplicon pooling and sequencing.

TABLE 1
Structure of the coding region of each KIR gene and lengths of the coding region,
non-coding region and genomic full-length sequence

| | Leader | | | | | Stem | Transmembrane region | | | | Non- | Genomic |
|---|---|---|---|---|---|---|---|---|---|---|---|---|
| | peptide | | Extra-cellulardomain | | | domain | and intracellulardomain | | | Coding | coding | full-length |
| KIR | Exon1 | Exon2 | Exon3 | Exon4 | Exon5 | Exon6 | Exon7 | Exon8 | Exon9 | sequence | sequence | sequence |
| gene | (bp) | (bp) | (bp) | (bp) | (bp) | (bp) | (bp) | (bp) | (bp) | (bp) | (bp) | (bp) |
| 2DL1 | 34 | 36 | Pseudoexon | 300 | 294 | 51 | 102 | 53 | 177 | 1047 | 13691 | 14738 |
| 2DL2 | 34 | 36 | Pseudoexon | 300 | 294 | 51 | 102 | 53 | 177 | 1047 | 13765 | 14812 |
| 2DL3 | 34 | 36 | Pseudoexon | 300 | 294 | 51 | 105 | 53 | 177 | 1050 | 13711 | 14761 |
| 2DL4 | 40 | 36 | 285 | Del | 284 | 51 | 105 | 53 | 270 | 1134 | 10043 | 11177 |
| 2DL5 | 34 | 36 | 285 | Del | 294 | 51 | 105 | 53 | 270 | 1128 | 8773 | 9901 |
| 2DS1 | 34 | 36 | Pseudoexon | 300 | 294 | 51 | 105 | 53 | 42 | 915 | 13805 | 14720 |
| 2DS2 | 34 | 36 | Pseudoexon | 300 | 294 | 51 | 105 | 53 | 42 | 915 | 13662 | 14377 |
| 2DS3 | 34 | 36 | Pseudoexon | 300 | 294 | 51 | 105 | 53 | 42 | 915 | 14189 | 15104 |
| 2DS4 | 34 | 36 | Pseudoexon | 300 | 294 | 51 | 105 | 53 | 42 | 915 | 15177 | 16092 |
| 2DS5 | 34 | 36 | Pseudoexon | 300 | 294 | 51 | 105 | 53 | 42 | 915 | 14333 | 15248 |
| 3DL1 | 34 | 36 | 285 | 300 | 294 | 51 | 105 | 53 | 177 | 1335 | 13211 | 14546 |
| 3DL2 | 34 | 36 | 285 | 300 | 294 | 51 | 105 | 53 | 210 | 1368 | 15641 | 17009 |
| 3DL3 | 34 | 36 | 285 | 300 | 294 | del | 105 | 53 | 126 | 1233 | 11182 | 12415 |
| 3DS1 | 34 | 36 | 285 | 300 | 294 | 51 | 105 | 51 | 8 | 1164 | 13980 | 15144 |
| 2DP1 | 34 | 36 | Pseudoexon | 299 | 294 | 51 | 105 | 53 | 177 | 1331 | / | 13126 |
| 3DP1 | 34 | Pseudoexon | 285 | 300 | 294 | / | / | / | / | / | / | 5713 |

Note:
2DP1 and 3DP1 in this table are Pseudogenes; Del: exon deletion; Pseudoexon: pseudoexon.

The structure of KIR genes is complex. Among KIR2D genes, exon 3 of 8 KIR genes including KIR2DL1-3 and 2DS1-5 is a pseudoexon, which cannot encode the corresponding DO extra-cellular domain. Both KIR2DL4 and 2DL5 have a deletion of exon 4 and cannot encode the D1 extra-cellular domain. Among functional KIR3D genes, except that KIR3DL3 has a deletion of exon 6, the other functional KIR3D genes (3DL1, 3DL2 and 3DS1) all contain 9 exons, 8 introns, a 5′-promoter region, and a 3′-UTR region.

KIR genes are inherited in haplotypes with complex molecular genetic polymorphisms, as evidenced by: (1) Diversity in the composition and copy number of KIR genes on KIR haplotypes: Generally, except for 4 framework genes (3DL3, 3DP1, 2DL4, and 3DL2), other KIR genes may be present or absent in individuals, with the copy number of each gene of 0-3; (2) Allelic level: As of December 2024, the total number of KIR alleles released in the international IPD-KIR Database (Release 2.13) has reached 2,219 (including 47 null alleles); each KIR gene has 33-428 different alleles (see Table 2 for details). The homology between the sequences of any two different KIR alleles is as high as 85-98%^{[3]}. The genetic complexity of the KIR gene cluster and the high homology of allele sequences pose great challenges to sequencing-based typing of KIR genes.

TABLE 2
KIR alleles released in the IPD-KIR database (Release 2.13)

| | Number of alleles with | Number of |
|---|---|---|
| Gene | published sequences | null alleles |
| 2DL1 | 428 | 9 |
| 2DL2 | 35 | 0 |
| 2DL3 | 68 | 1 |
| 2DL4 | 114 | 0 |
| 2DL5A | 45 | 1 |
| 2DL5B | 47 | 0 |
| 2DS1 | 33 | 0 |
| 2DS2 | 65 | 0 |
| 2DS3 | 71 | 2 |
| 2DS4 | 41 | 22 |
| 2DS5 | 88 | 0 |
| 3DL1 | 307 | 5 |
| 3DL2 | 252 | 4 |
| 3DL3 | 232 | 1 |
| 3DS1 | 91 | 2 |
| 2DP1 | 114 | 0 |
| 3DP1 | 188 | 0 |
| Total | 2219 | 47 |

The identification of KIR alleles has functional significance. The expression levels of different KIR genes are different^{[4]}, and the expression levels, affinities, and mediated activation/inhibition capacities of different alleles in the same KIR gene are also significantly different. For example, the expression levels of 3DL1 alleles on the surface of NK cells, from highest to lowest, are KIR3DL1*01502>*020>*001>*007>*005, and the strengths of the mediated inhibitory effects thereof, in order, are KIR3DL1*001>*005>*01502>*020 >*007^{[5]}.

The identification of KIR alleles has clinical significance. Boudreau et al. analyzed 1,328 patients with acute myeloid leukemia who received hematopoietic stem cell transplantation from HLA 9-10/10 matched unrelated donors. Donors carrying non-expressed (null) or low-expression alleles of 3DL1 could exert an anti-leukemic effect and improve overall survival^{[6]}. Dubreuil et al. found through research that individuals with the centromeric KIR AA haplotype group (cenAA) tend to carry high-expression alleles 2DL1*003 and 2DL3*001. Their NK cell clones exhibit higher expression levels of the corresponding KIRs and stronger NK cell activity; donor-derived cenAA can significantly reduce the relapse of myeloid leukemia^{[7]}.

Due to the functional and clinical significance of the identification of KIR alleles, it is particularly important to create a high-throughput and high-resolution sequencing-based typing technology for full-length sequences of KIR genes and conduct KIR application research at the allele level. However, existing methods for detecting polymorphisms of KIR genes at home and abroad are not suitable for sequencing-based typing of full-length sequences of KIR genes. The reasons are as follows:
(I) The widely used clinical methods including polymerase chain reaction-sequence specific primers (PCR-SSP), flow beads-reverse sequence-specific oligonucleotide probes (Flow-rSSO) and quantitative PCR (Q-PCR), and commercially available kits can only identify the presence or absence of each KIR gene at low-resolution level. These methods cannot identify allelic polymorphisms at high-resolution level, nor can they distinguish all non-expressed KIR alleles and variants^{[8, 9]}.
(II) Based on the Sanger sequencing-based typing technique, the applicant has analyzed the existed problems of KIR gene sequencing-based typing (PCR-SBT) methods reported at home and abroad^{[10]}: (1) Only partial exons of KIR genes were subjected to sequencing-based typing^{[11, 12, 13]}. (2) The fragments of PCR product and the required amplification time were excessively long^{[14]}. (3) There is a phenomenon of non-specific amplification. For example, if the subject carries both the 2DL1 and 2DS1 genes, while amplifying the sequences of exons 1-5 of 2DL1, the sequences of the highly homologous 2DS1 gene are also co-amplified^{[15]}. (4) Simultaneous PCR amplification and sequencing for all KIR genes cannot be performed under the same cycling parameters^{[14, 15, 16]}, making high-throughput analysis impossible and the experimental operation time-consuming and labor-intensive^{[10]}.

In order to solve the above problems, the applicant has developed a scientific PCR amplification strategy based on the structure of the genomic full-length sequence of each KIR gene, the distribution characteristics of single nucleotide polymorphisms (SNPs) in the coding regions, and the lengths of introns flanking the exons. They designed and adopted KIR gene-specific PCR primers with similar annealing temperatures for simultaneous PCR amplification, thereby establishing a method for use in simultaneous sequencing-based typing of 14 functional killer immunoglobulin-like receptor (KIR) genes^{[17, 18]}.

However, due to the inherent limitations of Sanger sequencing technology, the Sanger sequencing-based typing methods for KIR genes available at home and abroad^{[11-18]} have the following shortcomings: First, there are 16 KIR genes, and each exon of each KIR gene carried by the subject needs to be sequenced in the forward and reverse directions, which is time-consuming and labor-intensive; second, sequencing-based typing can only cover the exonic region of each KIR gene, not the non-coding region; third, the more KIR alleles, the more likely ambiguous genotyping results; fourth, although KIR alleles can be identified at high resolution level, the copy number of KIR alleles cannot be determined.

(III) Next generation sequencing (NGS) sequencing methods for KIR genes based on capture technology or PCR amplification^{[19, 20]}, due to the short read lengths (100-300 bp) of second-generation sequencing, and highly homologous full-length sequences of KIR genes (full-length sequences of functional KIR genes in IPD-KIR database are 9901-17,009 bp), face the risk of incomplete sequence assembly and failed sequencing-based typing after library construction and sequencing^{[20]}. These methods require inferring KIR genotypes through cumbersome and complex processes, including expectation-maximization algorithms, linkage inheritance analysis of KIR alleles, and bioinformatics analysis. However, the accuracy of the results is difficult to guarantee. To date, there is no commercially available NGS KIR gene sequencing-based typing kit at home and abroad.

Third-generation sequencing technology, a single-molecule sequencing technology, has the advantage of long read lengths (10-100 kb). When applied to full-length sequence determination and genotyping of KIR genes, an amplification-based library construction method is adopted, which eliminates the need for fragmenting PCR products, with no ambiguity in the results. Compared with Sanger and NGS KIR gene sequencing-based typing technologies, the third-generation sequencing technology has significant technical advantages. To date, however, there is no mature KIR gene third-generation long-read sequencing-based typing technology and no related commercially available kits.

With the in-depth advancement of research on the biological functions of KIR molecules, disease association and hematopoietic stem cell transplantation, the application and research fields of KIR have become increasingly extensive. For the KIR gene cluster with complex structures and genetic polymorphisms and high sequence homology, establishing a high-efficiency, low-cost and high-throughput third-generation long-read sequencing-based typing technology and achieving commercialization and industrialization thereof help break through the technical bottlenecks in KIR gene sequencing-based typing at home and abroad. This is a key technical problem that urgently needs to be solved in the field of molecular immunogenetics and transplant immunology.

### REFERENCES

[1] Vierra-Green C, Roe D, Hou L, et al. Allele-Level Haplotype Frequencies and Pairwise Linkage Disequilibrium for 14 KIR Loci in 506 European-American Individuals. PLOS One, 2012, 7(11): e47491.
[2] Robinson J, Mistry K, McWilliam H, et al. IPD—the Immuno Polymorphism Database. Nucleic Acids Res. 2010, 38: D863-869.
[3] Jiang W, Johnson C, Simecek N, et al. qKAT: a High-Throughput qPCR Method for KIR Gene Copy Number and Haplotype Determination. Genome Med. 2016, 8(1): 99.
[4] McErlean C, Gonzalez A A, Cunningham R, et al. Differential RNA Expression of KIR Alleles. Immunogenetics, 2010, 62(7): 431-440.
[5] Yawata M, Yawata N, Draghi M, et al. Roles for HLA and KIR Polymorphisms in Natural Killer Cell Repertoire Selection and Modulation of Effector Function. J Exp Med, 2006, 203(3): 633-645.
[6] Boudreau J E, Giglio F, Gooley T A, et al. KIR3DL1/HLA-B Subtypes Govern Acute Myelogenous Leukemia Relapse After Hematopoietic Cell Transplantation. J Clin Oncol. 2017, 35(20): 2268-2278.
[7] Dubreuil L, Maniangou B, Chevallier P, et al. Centromeric KIR AA Individuals Harbor Particular KIR Alleles Conferring Beneficial NK Cell Features with Implications in Haplo-Identical Hematopoietic Stem Cell Transplantation. Cancers (Basel). 2020, 12(12): 3595.
[8] Deng Zhihui, Zhen Jianxin, Zhang Geng, Yang Zhichao, Yu Qiong, Chen Hao. Establishment of a PCR-SSP Method for the Simultaneous Amplification and Identification of the Presence or Absence of KIR Genes. Chinese Journal of Medical Genetics. 2023, 40(7): 881-886.
[9] Li Yunnan, Zhen Jianxin, Liang Shuang, Yu Qiong, Deng Zhihui. Establishment of Q-PCR Method for Qualitative Testing of the Presence or Absence of KIR Genes. China Journal of Blood Transfusion. 2024, 37(6): 660-665.
[10] Zhen Jianxin, Yu Qiong. Progress in Research on Genetic Polymorphisms and Sequence-based Typing of KIR Genes. Chinese Journal of Medical Genetics. 2016, 33(6): 867-870.
[11] Lebedeva T V, Ohashi M, Zannelli G, et al. Comprehensive Approach to High-resolution KIR Typing. Hum Immunol, 2007, 68(9): 789-796.
[12] Yan L X, Zhu F M, Jiang K, et al. Diversity of the Killer Cell Immunoglobulin-like Receptor Gene KIR2DS4 in the Chinese Population. Tissue Antigens, 2007, 69(2): 133-138.
[13] Buhler S, Di Cristofaro J, Frassati C, et al. High Levels of Molecular Polymorphism at the KIR2DL4 Locus in French and Congolese Populations: Impact for Anthropology and Clinical Studies. Hum Immunol. 2009, 70(11): 953-959.
[14] Belle I, Hou L, Chen M, et al. Investigation of Killer Cell Immunoglobulin-like Receptor Gene Diversity in KIR3DL1 and KIR3DS1 in a Transplant Population, Tissue Antigens. 2008, 71(5): 434-439.
[15] Hou L, Chen M, Steiner N, et al. Killer Cell Immunoglobulin-like Receptors (KIR) Typing by DNA Sequencing. Methods Mol Biol, 2012, 882:431-468.
[16] Meenagh A, Gonzalez A, Sleator C, et al. Investigation of Killer Cell Immunoglobulin-like Receptor Gene Diversity, KIR2DL1 and KIR2DS1. Tissue Antigens. 2008, 72(4): 383-391.
[17] Deng Zhihui, Zhen Jianxin, Zhang Guobin. Method for Use in Simultaneously Sequencing and Genotyping 14 Functional Killer Immunoglobulin-like Receptor (KIR) Genes, 2018 Mar. 20, Chinese Invention Patent, ZL 201710284545.3.
[18] Zhihui Deng, Jianxin Zhen, Guobin Zhang. Method for Simultaneous Sequence-based Typing of 14 Functional Killer Cell Immunoglobulin-like Receptor (KIR) Genes, 2019 Apr. 23, U.S. Pat. No. 10,266,877 B2.
[19] Norman P J, Hollenbach J A, Nemat-Gorgani N, et al. Defining KIR and HLA Class I Genotypes at Highest Resolution via High-Throughput Sequencing. Am J Hum Genet. 2016, 99(2): 375-391.
[20] Wagner I, Schefzyk D, Pruschke J, et al. Allele-Level KIR Genotyping of More Than a Million Samples: Workflow, Algorithm, and Observations. Front Immunol. 2018; 9:2843.

### SUMMARY

Aiming at breaking through the technical bottlenecks present in KIR gene sequencing-based typing at home and abroad, the present disclosure for the first time provides a method for genotyping of all 16 killer immunoglobulin-like receptor (KIR) genes based on third-generation long-read sequencing, which can be applied to research fields such as KIR population genetics, molecular evolution, donor-recipient histocompatibility matching in hematopoietic stem cell transplantation, and disease association studies, laying a foundation for the commercialization and industrialization of KIR gene sequencing-based typing reagents.

The present disclosure proposes a PCR primer set for amplifying full-length sequences of 16 KIR genes, which primer set includes 3 pairs of specific PCR primers, where the 3 pairs of specific PCR primers include a first PCR primer pair, a second PCR primer pair and a third PCR primer pair;

sequences of the first PCR primer pair are set forth in SEQ ID NOs. 1-2, sequences of the second PCR primer pair are set forth in SEQ ID NOs. 3-4, and sequences of the third PCR primer pair are set forth in SEQ ID NOs. 5-6;

the first PCR primer pair is used for specific multiplex PCR amplification of the full-length sequences of the 2DL1, 2DL2, 2DL3, 2DL5, 2DS1, 2DS2, 2DS3, 2DS4, 2DS5, 3DL1, 3DL2, 3DL3, 3DS1 and 2DP1 genes, the second PCR primer pair is used for specific amplification of the full-length sequence of the 2DL4 gene, and the third PCR primer pair is used for specific amplification of the full-length sequence of the 3DP1 gene and a partial sequence of one KIR gene closest to the 3DP1 gene in an upstream flanking region thereof.

In one embodiment, in the third PCR primer pair used for specific amplification of the full-length sequence of the 3DP1 gene and a partial sequence of one KIR gene closest to the 3DP1 gene in an upstream flanking region thereof, the one KIR gene closest to the 3DP1 gene in the upstream flanking region thereof includes any one of the 2DL1, 2DL2, 2DL3, 2DL5, 2DS2, 2DS3, 2DS5, 3DL3 and 2DP1 genes; and the partial sequence of the one KIR gene closest to the 3DP1 gene includes a sequence from intron 5 to 3′-untranslated region of the KIR gene closest to the 3DP1 gene.

In one embodiment, the PCR amplification primer set is used for amplification of the full-length sequences of all 16 KIR genes in humans by long-range PCR method and library construction in third-generation long-read sequencing-based typing of KIR genes; and lengths of PCR products of the first PCR primer pair, the second PCR primer pair and the third PCR primer pair range from 9.2 kb to 16.7 kb, covering all exons and introns of each KIR gene.

The present disclosure further provides a PCR amplification kit for amplifying full-length sequences of 16 KIR genes, which kit includes the PCR amplification primer set for amplifying full-length sequences of 16 KIR genes described above and PCR amplification reagents.

The present disclosure further provides a method for genotyping 16 KIR genes based on third-generation long-read sequencing, which method includes steps of:
1) mixing a first PCR primer pair, a second PCR primer pair and a third PCR primer pair separately with genomic DNA of a test sample, DNA polymerase mixture for rapid amplification of long fragment and ddH₂O to prepare PCR amplification reaction systems, thereby obtaining a first PCR amplification reaction system, a second PCR amplification reaction system and a third PCR amplification reaction system, where sequences of the first PCR primer pair are set forth in SEQ ID NOs. 1-2, sequences of the second PCR primer pair are set forth in SEQ ID NOs. 3-4, and sequences of the third PCR primer pair are set forth in SEQ ID NOs. 5-6;
2) subjecting the first PCR amplification reaction system, the second PCR amplification reaction system and the third PCR amplification reaction system to amplification respectively under corresponding PCR conditions to obtain a first amplification product, a second amplification product and a third amplification product, and mixing the three amplification products into one tube at a mass ratio of 30:2:3 to obtain a pooled PCR amplification product; and
3) subjecting the pooled PCR amplification product to magnetic bead purification, concentration and fragment size detection, followed by library construction, on-machine library preparation, on-machine sequencing and bioinformatics analysis, whereby the KIR genotype of the test sample can be determined.

In one embodiment, the PCR amplification reaction systems are respectively as follows:
the first PCR amplification reaction system is 25.0 μL in volume and includes components of: 2.0 μL of 10 μM the first PCR primer pair, 2.0 μL of 50-100 ng/μL genomic DNA, 12.5 μL of DNA polymerase mixture for rapid amplification of long-fragment, and 8.5 μL of ddH₂O;
the second PCR amplification reaction system is 25.0 μL in volume and includes components of: 2.0 μL of 10 μM the second PCR primer pair, 2.0 μL of 50-100 ng/μL genomic DNA, 12.5 μL of DNA polymerase mixture for rapid amplification of long-fragment, and 8.5 μL of ddH₂O; and
the third PCR amplification reaction system is 25.0 μL in volume and includes components of: 6.0 μL of 1.0 μM the third PCR primer pair, 2.0 μL of 50-100 ng/μL genomic DNA, 12.5 μL of DNA polymerase mixture for rapid amplification of long-fragment, and 4.5 μL of ddH₂O.

In one embodiment, conditions for the PCR amplification are respectively as follows: reaction program for the first PCR amplification reaction system are: 98° C. for 2 min; 98° C. for 10 Sec; 59° C. for 10 Sec; 72° C. for 4 min, for 35 cycles; and 72° C. for 5 min; reaction program for the second PCR amplification reaction system are: 98° C. for 2 min; 98° C. for 10 Sec; 56° C. for 10 Sec; 72° C. for 4 min, for 35 cycles; and 72° C. for 5 min; and reaction program for the third PCR amplification reaction system are: 98° C. for 2 min; 98° C. for 10 Sec; 65° C. for 10 Sec; 72° C. for 5 min, for 35 cycles; and 72° C. for 5 min.

In one embodiment, lengths of amplification products of the first PCR primer pair, the second PCR primer pair and the third PCR primer pair range from 9.2 kb to 16.7 kb, covering all exons and introns of each KIR gene.

The contributions of the present disclosure lie in that: A high-throughput third-generation long-read sequencing-based typing method for KIR genes is established for the first time. Based on the polymorphisms of KIR allele sequences in Chinese population, and the structure of full-length sequences of KIR genes and the distribution of single nucleotide polymorphisms released in the IPD-KIR database, 3 pairs of specific primers are designed for all 16 KIR genes in the KIR gene cluster, and the full-length sequences of all 16 KIR genes in humans are amplified by long-range PCR, as shown in FIG. 1. The amplification products, after being determined for concentration and purity, are mixed into one tube at a certain mass ratio, and subjected to library construction, on-machine sequencing (PacBio platform) and bioinformatics analysis, whereby KIR alleles carried by a subject can be determined.

The technical advantages of the present disclosure lie in that: The full-length sequences of all 16 KIR genes can be amplified via only 3 separate PCR reactions, and then mixing the three PCR amplification products into one tube at a certain mass ratio for third-generation sequencing. The method has the characteristics of simple experimental operation for amplification and library construction, low cost, genotyping covering the full-length sequence of each KIR gene, no ambiguous results, and high quality of the obtained sequences. Compared with Sanger and NGS-based KIR gene sequencing methods, the method of the present disclosure has significant technical advantages, which can lay a foundation for the subsequent commercialization and industrialization of the technology, and thus has broad application prospects in fields such as KIR population genetics, disease association and hematopoietic stem cell transplantation.

### BRIEF DESCRIPTION OF THE DRAWINGS

FIG. 1 shows PCR amplification strategies for amplifying full-length sequences of 16 KIR genes;

FIG. 2 shows an agarose gel electrophoresis image of the PCR amplification products of the sample of Example 1 (No. 13013519, KIR AA1 genotype);

FIG. 3 shows a visualization diagram of the alignment of target sequences for all 9 KIR genes carried by the sample of Example 1 with the reference genome;

FIG. 4 shows an agarose gel electrophoresis image of the PCR amplification products of the sample of Example 2 (No. 13013484, KIR AB8 genotype); and

FIG. 5 shows a visualization diagram of the alignment of target sequences for all 13 KIR genes carried by the sample of Example 2 with the reference genome.

### DETAILED DESCRIPTION OF THE EMBODIMENTS

In order to make the objects, technical solutions and advantages of the present disclosure clearer, the technical solutions in the embodiments of the present disclosure will be clearly and completely described below. Apparently, the described embodiments are merely some, rather than all of the embodiments of the present disclosure.

Due to the functional and clinical significance of the identification of KIR alleles, it is particularly important to create a high-throughput and high-resolution sequencing-based typing technology for full-length sequences of KIR genes and conduct KIR application research at the allele level. However, existing methods for detecting polymorphisms of KIR genes at home and abroad are not suitable for sequencing-based typing of full-length sequences of KIR genes.

Third-generation sequencing technology, a single-molecule sequencing technology, has the advantage of long read lengths (10-100 kb). When applied to full-length sequence determination and genotyping of KIR genes, an amplification-based library construction method is adopted, which eliminates the need for fragmenting PCR products, with no ambiguity in the results. Compared with Sanger and NGS KIR gene sequencing-based typing technologies, the third-generation sequencing technology has significant technical advantages. To date, however, there is no mature KIR gene third-generation long-read sequencing-based typing technology and no related commercially available kits.

With the in-depth advancement of research on the biological functions of KIR molecules, disease association and hematopoietic stem cell transplantation, the application and research fields of KIR have become increasingly extensive. For the KIR gene cluster with complex structures and genetic polymorphisms and high sequence homology, establishing a high-efficiency, low-cost and high-throughput third-generation long-read sequencing-based typing technology and achieving commercialization and industrialization thereof help break through the technical bottlenecks in KIR gene sequencing-based typing at home and abroad. This is a key technical problem that urgently needs to be solved in the field of molecular immunogenetics and transplant immunology.

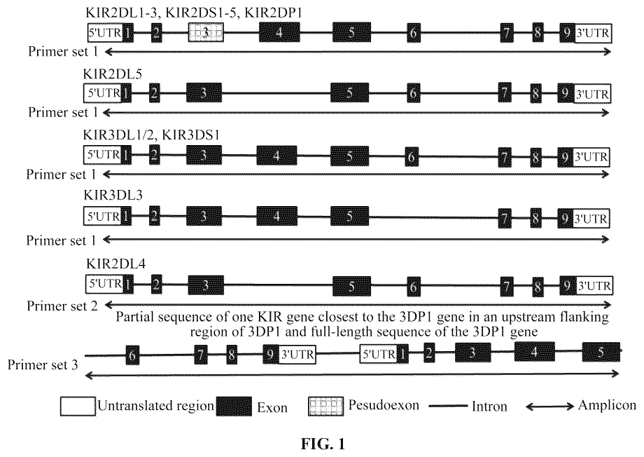

In view of this, the present disclosure proposes a “method for genotyping 16 KIR genes based on third-generation long-read sequencing”, which includes a set of 3 pairs of specific PCR amplification primers. The characteristics such as the sequences of each PCR primer pair, the positions of the primers in the genomic full-length sequences of KIR genes, and the lengths of the target amplicons are described in Table 3, Table 4 and Table 5. The full-length sequences of all 16 KIR genes in humans are amplified by long-range PCR. The first PCR primer pair is used for specific multiplex PCR amplification of the full-length sequences of 14 KIR genes (including 2DL1, 2DL2, 2DL3, 2DL5, 2DS1, 2DS2, 2DS3, 2DS4, 2DS5, 3DL1, 3DL2, 3DL3, 3DS1 and 2DP1), excluding 2DL4 and 3DP1. The second PCR primer pair is used for specific amplification of the full-length sequence of the 2DL4 gene. The third PCR primer pair is used for specific amplification of the full-length sequence of the 3 DPI gene and a partial sequence of one KIR gene closest to the 3DP1 gene in an upstream flanking region thereof. See FIG. 1 for details of the PCR amplification strategies.

TABLE 3
Positions of the first PCR primer pair to bind to each KIR gene, located regions of the primers and
lengths of amplification regions of the primers and lengths of amplification products

| | | Forward primer sequence and | | | Reverse primer sequences and | | | |
|---|---|---|---|---|---|---|---|---|
| | | characteristics thereof | | | characteristics thereof | | | Length |
| | | | Position in the | | | Position in the | | of ampli- |
| | | Primer | full-length | | Primer | full-length | | fication |
| | KIR | sequence | sequence of | Located | sequence | sequence of | Located | product |
| No. | gene | (5′→3′) | each KIR gene | region | (5′→3′) | each KIR gene | region | (bp) |
| 1 | 2DL1 | AAKAGCCTGCRT | nt-129 to nt-107 | 5′-UTR | GTTGGAGAGGTG | nt14228 to nt14206 | 3′-UTR | 14357 |
| 2 | 2DL2 | ACKTCAYCCTC | nt-129 to nt-107 | 5′-UTR | GGCAGGGGTCA | nt14270 to nt14248 | 3′-UTR | 14399 |
| 3 | 2DL3 | | nt-129 to nt-107 | 5′-UTR | | nt14251 to nt14229 | 3′-UTR | 14380 |
| 4 | 2DL5 | | nt-129 to nt-107 | 5′-UTR | | nt9168 to nt9146 | 3′-UTR | 9297 |
| 5 | 2DS1 | | nt-129 to nt-107 | 5′-UTR | | nt14232 to nt14210 | 3′-UTR | 14361 |
| 6 | 2DS2 | | nt-129 to nt-107 | 5′-UTR | | nt14056 to nt14034 | 3′-UTR | 14185 |
| 7 | 2DS3 | | nt-130 to nt-108 | 5′-UTR | | nt14562 to nt14540 | 3′-UTR | 14692 |
| 8 | 2DS4 | | nt-129 to nt-107 | 5′-UTR | | nt15604 to nt15582 | 3′-UTR | 15733 |
| 9 | 2DS5 | | nt-130 to nt-108 | 5′-UTR | | nt14738 to nt14716 | 3′-UTR | 14868 |
| 10 | 3DL1 | | nt-129 to nt-107 | 5′-UTR | | nt14039 to nt14017 | 3′-UTR | 14168 |
| 11 | 3DL2 | | nt-129 to nt-107 | 5′-UTR | | nt16491 to nt16469 | 3′-UTR | 16620 |
| 12 | 3DL3 | | nt-124 to nt-102 | 5′-UTR | | nt11873 to nt11851 | 3′-UTR | 11997 |
| 13 | 3DS1 | | nt-129 to nt-107 | 5′-UTR | | nt14423 to nt14401 | 3′-UTR | 14552 |
| 14 | 2DP1 | | nt-129 to nt-107 | 5′-UTR | | nt12619 to nt12597 | 3′-UTR | 12748 |

TABLE 4
Positions of the first PCR primer pair to bind to each KIR gene, located regions
of the primers and lengths of amplification products

| | | Forward primer sequence and | | | Reverse primer sequences and | | | Length |
|---|---|---|---|---|---|---|---|---|
| | | characteristics thereof | | | characteristics thereof | | | of |
| | | | Position in the | | | Position in the | | ampli- |
| | | Primer | full-length | | Primer | full-length | | fication |
| | KIR | sequence | sequence | Located | sequence | sequence | Located | product |
| No. | gene | (5′→3′) | of each KIR gene | region | (5′→3′) | of each KIR gene | region | (bp) |
| 1 | 2DL4 | CRTTGCGCATGA | nt-228 to | 5′-UTR | ATGRRGGSAGACAT | nt10910 to | 3′-UTR | 11138 |
| | | TGTGAASTGAC | nt-2067 | | GTTTAYTTGAA | nt10886 | | |

TABLE 5
Positions of the third PCR primer pair to bind to each KIR gene, located regions

| of the primers and lengths of amplification products | | | | | | | | |
|---|---|---|---|---|---|---|---|---|
| | | Forward primer sequence and | | | Reverse primer sequences and | | | Length |
| | | characteristics thereof | | | characteristics thereof | | | of |
| | | | Position in the | | | Position in the | | ampli- |
| | | Primer | full-length | | Primer | full-length | | fication |
| | KIR | sequence | sequence of | Located | sequence | sequence of | Located | product |
| No. | gene | (5′→3′) | each KIR gene | region | (5′→3′) | each KIR gene | region | (bp) |
| 1 | 2DL1- | ACASRGCACCTC | nt5753~nt5775 | 2DL1 | ACAAGCAGTGGGT | nt5667~nt5645 | 3DP1 | 16405 |
| | 3DP1 | SAARCCCTCYT | | Intron 5 | CACTAAGGTC | | Exon 5 | |
| 2 | 2DL2- | | nt5670~nt5692 | 2DL2 | | nt5667~nt5645 | 3DP1 | 16518 |
| | 3DP1 | | | Intron 5 | | | Exon 5 | |
| 3 | 2DL3- | | nt5672~nt5694 | 2DL3 | | nt5667~nt5645 | 3DP1 | 16509 |
| | 3DP1 | | | Intron 5 | | | Exon 5 | |
| 4 | 2DL5- | | nt3166~nt3188 | 2DL5 | | nt5667~nt5645 | 3DP1 | 13932 |
| | 3DP1 | | | Intron 5 | | | Exon 5 | |
| 5 | 2DS2- | | nt5558~nt5580 | 2DS2 | | nt5667~1115645 | 3DP1 | 16407 |
| | 3DP1 | | | Intron 5 | | | Exon 5 | |
| 6 | 2DS3- | | nt6066~4116088 | 2DS3 | | nt5667~nt5645 | 3DP1 | 16426 |
| | 3DP1 | | | Intron 5 | | | Exon 5 | |
| 7 | 2DS5- | | nt6260~nt6282 | 2DS5 | | nt5667~nt5645 | 3DP1 | 16408 |
| | 3DP1 | | | Intron 5 | | | Exon 5 | |
| 8 | 3DL3- | | n15397~nt5419 | 3DL3 | | nt5667~415645 | 3DP1 | 14385 |
| | 3DP1 | | | Intron | | | Exon 5 | |
| | | | | 5/6 | | | | |
| 9 | 2DP1- | | nt5851~nt5873 | 2DP1 | | nt5667~nt5645 | 3DP1 | 14698 |
| | 3DP1 | | | Intron 5 | | | Exon 5 | |

The sequences of the 3 pairs of specific PCR primers, the positions of the primers in the genomic full-length sequence of each KIR gene, the lengths of the target amplicons, and the covered regions are specifically as follows:

1) First PCR primer pair
Forward primer sequence:
(SEQ ID NO. 1)
5′-AAKAGCCTGCRTACKTCAYCCTC-3′;
and
reverse primer sequence:
(SEQ ID NO. 2)
5′-GTTGGAGAGGTGGGCAGGGGTCA-3′.

The primer pair is used for amplification of the genomic full-length sequences of 14 KIR genes (including 2DL1, 2DL2, 2DL3, 2DL5, 2DS1, 2DS2, 2DS3, 2DS4, 2DS5, 3DL1, 3DL2, 3DL3, 3DS1, and 2DP1), excluding 2DL4 and 3DP1. The characteristics such as the positions of the primer pair in the genomic full-length sequences of the 14 KIR genes, and the lengths of the target amplicons are as follows:
i) For the 2DL1 gene: The position of the forward primer in the genomic full-length sequence of the 2DL1 gene is nt-129 to nt-107, in the 5′-UTR region; the position of the reverse primer in the genomic full-length sequence of the 2DL1 gene is nt14228 to nt14206, in the 3′-UTR region; the length of the target amplicon is 14,357 bp, covering all exons and all introns of the 2DL1 gene.
ii) For the 2DL2 gene: The position of the forward primer in the genomic full-length sequence of the 2DL2 gene is nt-129 to nt-107, in the 5′-UTR region; the position of the reverse primer in the genomic full-length sequence of the 2DL2 gene is nt14270 to nt14248, in the 3′-UTR region; the length of the target amplicon is 14,399 bp, covering all exons and all introns of the 2DL2 gene.
iii) For the 2DL3 gene: The position of the forward primer in the genomic full-length sequence of the 2DL3 gene is nt-129 to nt-107, in the 5′-UTR region; the position of the reverse primer in the genomic full-length sequence of the 2DL3 gene is nt14251 to nt14229, in the 3′-UTR region; the length of the target amplicon is 14,380 bp, covering all exons and all introns of the 2DL3 gene.
iv) For the 2DL5 gene: The position of the forward primer in the genomic full-length sequence of the 2DL5 gene is nt-129 to nt-107, in the 5′-UTR region; the position of the reverse primer in the genomic full-length sequence of the 2DL5 gene is nt9168 to nt9146, in the 3′-UTR region; the length of the target amplicon is 9,297 bp, covering all exons and all introns of the 2DL5 gene.
v) For the 2DS1 gene: The position of the forward primer in the genomic full-length sequence of the 2DS1 gene is nt-129 to nt-107, in the 5′-UTR region; the position of the reverse primer in the genomic full-length sequence of the 2DS1 gene is nt14232 to nt14210, in the 3′-UTR region; the length of the target amplicon is 14,361 bp, covering all exons and all introns of the 2DS1 gene.
vi) For the 2DS2 gene: The position of the forward primer in the genomic full-length sequence of the 2DS2 gene is nt-129 to nt-107, in the 5′-UTR region; the position of the reverse primer in the genomic full-length sequence of the 2DS2 gene is nt14056 to nt14034, in the 3′-UTR region; the length of the target amplicon is 14,185 bp, covering all exons and all introns of the 2DS2 gene.
vii) For the 2DS3 gene: The position of the forward primer in the genomic full-length sequence of the 2DS3 gene is nt-130 to nt-108, in the 5′-UTR region; the position of the reverse primer in the genomic full-length sequence of the 2DS3 gene is nt14562 to nt14540, in the 3′-UTR region; the length of the target amplicon is 14,692 bp, covering all exons and all introns of the 2DS3 gene.
viii) For the 2DS4 gene: The position of the forward primer in the genomic full-length sequence of the 2DS4 gene is nt-129 to nt-107, in the 5′-UTR region; the position of the reverse primer in the genomic full-length sequence of the 2DS4 gene is nt15604 to nt15582, in the 3′-UTR region; the length of the target amplicon is 15,733 bp, covering all exons and all introns of the 2DS4 gene.
ix) For the 2DS5 gene: The position of the forward primer in the genomic full-length sequence of the 2DS5 gene is nt-130 to nt-108, in the 5′-UTR region; the position of the reverse primer in the genomic full-length sequence of the 2DS5 gene is nt14738 to nt14716, in the 3′-UTR region; the length of the target amplicon is 14,868 bp, covering all exons and all introns of the 2DS5 gene.
x) For the 3DL1 gene: The position of the forward primer in the genomic full-length sequence of the 3DL1 gene is nt-129 to nt-107, in the 5′-UTR region; the position of the reverse primer in the genomic full-length sequence of the 3DL1 gene is nt14039 to nt14017, in the 3′-UTR region; the length of the target amplicon is 14,168 bp, covering all exons and all introns of the 3DL1 gene.
xi) For the 3DL2 gene: The position of the forward primer in the genomic full-length sequence of the 3DL2 gene is nt-129 to nt-107, in the 5′-UTR region; the position of the reverse primer in the genomic full-length sequence of the 3DL2 gene is nt16491 to nt16469, in the 3′-UTR region; the length of the target amplicon is 16,620 bp, covering all exons and all introns of the 3DL2 gene.
xii) For the 3DL3 gene: The position of the forward primer in the genomic full-length sequence of the 3DL3 gene is nt-124 to nt-102, in the 5′-UTR region; the position of the reverse primer in the genomic full-length sequence of the 3DL3 gene is nt11873 to nt11851, in the 3′-UTR region; the length of the target amplicon is 11,997 bp, covering all exons and all introns of the 3DL3 gene.
xiii) For the 3DS1 gene: The position of the forward primer in the genomic full-length sequence of the 3DS1 gene is nt-129 to nt-107, in the 5′-UTR region; the position of the reverse primer in the genomic full-length sequence of the 3DS1 gene is nt14423 to nt14401, in the 3′-UTR region; the length of the target amplicon is 14,552 bp, covering all exons and all introns of the 3DS1 gene.
xiv) For the 2DP1 gene: The position of the forward primer in the genomic full-length sequence of the 2DP1 gene is nt-129 to nt-107, in the 5′-UTR region; the position of the reverse primer in the genomic full-length sequence of the 2DP1 gene is nt12619 to nt12597, in the 3′-UTR region; the length of the target amplicon is 12,748 bp, covering the full-length sequence of the 2DP1 gene from the 5′-UTR region to the 3′-UTR region.

2) Second PCR primer pair
Forward primer sequence:
(SEQ ID NO. 3)
and5′-CRTTGCGCATGATGTGAASTGAC-3′;
reverse primer sequence:
(SEQ ID NO. 4)
5′-ATGRRGGSAGACATGTTTAYTTGAA-3′.

The primer pair is used for specific amplification of the 2DL4 gene. The position of the forward primer in the genomic full-length sequence of the 2DL4 gene is nt-228 to nt-206, in the 5′-UTR region; the position of the reverse primer in the genomic full-length sequence of the 2DL4 gene is nt10910 to nt10886, in the 3′-UTR region; the length of the amplification fragment is 11,138 bp, covering all exons and all introns of the 2DL4 gene.

3) Third PCR primer pair
Forward primer sequence:
(SEQ ID NO. 5)
5′-ACASRGCACCTCSAARCCCTCYT-3′;
and
reverse primer sequence:
(SEQ ID NO. 6)
5′-ACAAGCAGTGGGTCACTAAGGTC-3′.

The primer pair is used for specific amplification of the full-length sequence of the 3DP1 gene and a partial sequence of the first KIR gene in an upstream flanking region thereof, and the full lengths of the PCR amplification products range from 13,932 bp to 16,518 bp (see Table 5 for details). The purpose of such amplification is to avoid preferential library construction that occurs when only the full-length sequence of the 3DP1 gene (5713 bp) is amplified due to the short fragment length thereof, which would otherwise affect the library construction and sequencing of target fragments of other KIR genes.

The positions of the primer pair in the full-length sequence of each KIR gene, the lengths of the target amplicons, and the covered regions are specifically as follows:
i) 2DL1-3DP1: If the first KIR gene in the upstream flanking region of the 3 DPI gene is the 2DL1 gene, the position of the forward primer in the genomic full-length sequence of the 2DL1 gene is nt5753 to nt5775, in intron 5; the position of the reverse primer in the genomic full-length sequence of the 3DP1 gene is nt5667 to nt5645, at the end of exon 5; the length of the target amplicon is 16,405 bp, covering a partial sequence of intron 5 and a sequence from exon 6 to the 3′-UTR region of the 2DL1 gene, an intergenic sequence between the 2DL1 and 3DP1 genes, and the full-length sequence of the 3DP1 gene.
ii) 2DL2-3DP1: If the first KIR gene in the upstream flanking region of the 3DP1 gene is the 2DL2 gene, the position of the forward primer in the genomic full-length sequence of the 2DL2 gene is nt5670 to nt5692, in intron 5; the position of the reverse primer in the genomic full-length sequence of the 3DP1 gene is nt5667 to nt5645, at the end of exon 5; the length of the target amplicon is 16,518 bp, covering a partial region of intron 5 and a sequence from exon 6 to the 3′-UTR region of the 2DL2 gene, an intergenic sequence between the 2DL2 and 3 DPI genes, and the full-length sequence of the 3DP1 gene.
iii) 2DL3-3DP1: If the first KIR gene in the upstream flanking region of the 3 DPI gene is the 2DL3 gene, the position of the forward primer in the genomic full-length sequence of the 2DL3 gene is nt5672 to nt5694, in intron 5; the position of the reverse primer in the genomic full-length sequence of the 3DP1 gene is nt5667 to nt5645, at the end of exon 5; the length of the target amplicon is 16,509 bp, covering a partial sequence of intron 5 and a sequence from exon 6 to the 3′-UTR region of the 2DL3 gene, an intergenic sequence between the 2DL3 and 3DP1 genes, and the full-length sequence of the 3DP1 gene.
iv) 2DL5-3DP1: If the first KIR gene in the upstream flanking region of the 3 DPI gene is the 2DL5 gene, the position of the forward primer in the genomic full-length sequence of the 2DL5 gene is nt3166 to nt3188, in intron 5; the position of the reverse primer in the genomic full-length sequence of the 3DP1 gene is nt5667 to nt5645, at the end of exon 5; the length of the target amplicon is 13,932 bp, covering a partial sequence of intron 5 and a sequence from exon 6 to the 3′-UTR region of the 2DL5 gene, an intergenic sequence between the 2DL5 and 3DP1 genes, and the full-length sequence of the 3DP1 gene.
v) 2DS2-3DP1: If the first KIR gene in the upstream flanking region of the 3 DPI gene is the 2DS2 gene, the position of the forward primer in the genomic full-length sequence of the 2DS2 gene is nt5558 to nt5580, in intron 5; the position of the reverse primer in the genomic full-length sequence of the 3DP1 gene is nt5667 to nt5645, at the end of exon 5; the length of the target amplicon is 16,407 bp, covering a partial sequence of intron 5 and a sequence from exon 6 to the 3′-UTR region of the 2DS2 gene, an intergenic sequence between the 2DS2 and 3DP1 genes, and the full-length sequence of the 3DP1 gene.
vi) 2DS3-3DP1: If the first KIR gene in the upstream flanking region of the 3DP1 gene is the 2DS3 gene, the position of the forward primer in the genomic full-length sequence of the 2DS3 gene is nt6066 to nt6088, in intron 5; the position of the reverse primer in the genomic full-length sequence of the 3DP1 gene is nt5667 to nt5645, at the end of exon 5; the length of the target amplicon is 16,426 bp, covering a partial sequence of intron 5 and a sequence from exon 6 to the 3′-UTR region of the 2DS3 gene, an intergenic sequence between the 2DS3 and 3DP1 genes, and the full-length sequence of the 3DP1 gene.
vii) 2DS5-3DP1: If the first KIR gene in the upstream flanking region of the 3 DPI gene is the 2DS5 gene, the position of the forward primer in the genomic full-length sequence of the 2DS5 gene is nt6260 to nt6282, in intron 5; the position of the reverse primer in the genomic full-length sequence of the 3DP1 gene is nt5667 to nt5645, at the end of exon 5; the length of the target amplicon is 16,408 bp, covering a partial sequence of intron 5 and a sequence from exon 6 to the 3′-UTR region of the 2DS5 gene, an intergenic sequence between the 2DS5 and 3DP1 genes, and the full-length sequence of the 3DP1 gene.
viii) 3DL3-3DP1: If the first KIR gene in the upstream flanking region of the 3DP1 gene is the 3DL3 gene, the position of the forward primer in the genomic full-length sequence of the 3DL3 gene is nt5397 to nt5419, in intron 5; the position of the reverse primer in the genomic full-length sequence of the 3DP1 gene is nt5667 to nt5645, at the end of exon 5; the length of the target amplicon is 14,385 bp, covering a partial sequence of intron 5/6 and a sequence from exon 7 to the 3′-UTR region of the 3DL3 gene, an intergenic sequence between the 3DL3 and 3DP1 genes, and the full-length sequence of the 3DP1 gene.
ix) 2DP1-3DP1: If the first KIR gene in the upstream flanking region of the 3 DPI gene is the 2DP1 gene, the position of the forward primer in the genomic full-length sequence of the 2DP1 gene is nt5851 to nt5873, in intron 5; the position of the reverse primer in the genomic full-length sequence of the 3DP1 gene is nt5667 to nt5645, at the end of exon 5; the length of the target amplicon is 14,698 bp, covering a partial sequence of intron 5 and a sequence from exon 6 to the 3′-UTR region of the 2DP1 gene, an intergenic sequence between the 2DP1 and 3DP1 genes, and the full-length sequence of the 3DP1 gene.

The lengths of the amplification products of the first PCR primer pair, the second PCR primer pair and the third PCR primer pair all range from 9.2 kb to 16.7 kb.

The PCR amplification primer set for the full-length sequences of 16 KIR genes of the present disclosure (including 3 pairs of KIR gene-specific primers) is designed based on the polymorphisms of KIR allele sequences in the Chinese population, and the structure of full-length sequences of KIR genes and the distribution of single nucleotide polymorphisms released in the international IPD-KIR database. This design is suitable for the Chinese population and, in the third-generation long-read sequencing-based typing of KIR genes, suitable for the specific amplification of the full-length sequences of 16 KIR genes and library construction.

In the present disclosure, the detection reagent for PCR amplification of the full-length sequences of the 16 KIR genes includes: specific PCR primer pairs, DNA polymerase mixture for rapid amplification of long-fragment, genomic DNA and ddH₂O.

The present disclosure provides a method for genotyping 16 KIR genes based on third-generation long-read sequencing, which method preferably includes the steps of:
1) mixing a first PCR primer pair, a second PCR primer pair and a third PCR primer pair separately with genomic DNA of the test sample, DNA polymerase mixture for rapid amplification of long-fragment and ddH₂O to prepare PCR amplification reaction systems, thereby obtaining a first PCR amplification reaction system, a second PCR amplification reaction system and a third PCR amplification reaction system;
2) subjecting the first PCR amplification reaction system, the second PCR amplification reaction system and the third PCR amplification reaction system to amplification respectively under corresponding PCR conditions to obtain a first amplification product, a second amplification product and a third amplification product, and mixing the three amplification products into one tube at a certain mass ratio (30:2:3) to obtain a pooled PCR amplification product; and
3) subjecting the pooled PCR amplification product to magnetic bead purification, concentration and fragment size detection, followed by library construction, on-machine library preparation, on-machine sequencing and bioinformatics analysis, whereby the KIR genotype of the test sample can be determined.

The technical advantages of the present disclosure lie in that: The full-length sequences of all 16 KIR genes can be amplified via only 3 separate PCR reactions, and then mixing the three PCR amplification products into one tube at a certain mass ratio for third-generation sequencing. The method has the characteristics of simple experimental operation for amplification and library construction, low cost, genotyping covering the full-length sequence of each KIR gene, no ambiguous results, and high quality of the obtained sequences. Compared with Sanger and NGS-based KIR gene sequencing methods, the method of the present disclosure has significant technical advantages.

The technical solutions of the present disclosure are described in further detail below in conjunction with specific examples. It should be understood that the following examples are merely illustrative of the present disclosure and are not intended to limit the present disclosure.

### Implementation Materials

1. DNA polymerase mixture for rapid amplification of long-fragment: purchased from Jiangsu Cowin Biotech Co., Ltd, with the reagent batch number: 30424.
2. Samples with known KIR AA1 and KIR AB8 profile: derived from voluntary blood donors in Shenzhen. These are residual blood samples (5 mL per person) after laboratory testing of the donors' peripheral blood, anticoagulated with 5% EDTA, and collected by the applicant during the previous National Natural Science Foundation of China General Program (Project No.: 81373158; completed). The applicant has previously performed qualitative detection of the presence or absence of KIR genes at the low-resolution level using the Q-PCR method.
3. Samples from 20 unrelated healthy individuals: derived from volunteer blood donors in Shenzhen. These are residual blood samples (5 mL per person) after laboratory testing of the donors' peripheral blood, anticoagulated with 5% EDTA, and collected by the applicant during the previous National Natural Science Foundation of China General Program (Project No.: 81373158; completed). The applicant has previously performed whole-genome sequencing on these samples, and conducted KIR genotyping after extracting KIR-related sequences.

### Example 1

A Method for Genotyping Full-Length Sequences of 16 KIR Genes Suitable for the Chinese Population Based on Third-Generation Long-Read Sequencing

In previous work, the applicant determined the sequences of all coding regions of 14 functional KIR genes in the Chinese population (n=306) using the independently innovated and established “Method for simultaneous sequence-based typing of 14 functional killer immunoglobulin-like receptor (KIR) genes” (Chinese Invention Patent No.: ZL 201710284545.3; US Invention Patent No.: U.S. Pat. No. 10,266,877 B2, see References and in the “BACKGROUND” section for details), and identified 116 KIR alleles^{[21, 22, 23]}. As of January 2022, the applicant has previously discovered and identified a total of 75 novel alleles named by the World Health Organization (WHO) in previous work (see Table 6 for details on the new KIR alleles). In the present disclosure, 3 pairs of specific PCR primers were designed for all 16 KIR genes in the KIR gene cluster, based on the polymorphisms of KIR allelic sequence in the Chinese population and with reference to the full-length sequences of the KIR genes released in the international IPD-KIR database. The full-length sequences of all 16 KIR genes in humans were amplified by long-range PCR, with the lengths of the amplification products ranging from 9.2 kb to 16.7 kb. The characteristics such as the sequence and position of each PCR primer and lengths of the target amplicons are shown in Table 3 to Table 5.

TABLE 6
Seventy five novel KIR alleles previously discovered and identified by the applicant,
named by the WHO

| | | | | | | Exon | | | |
|---|---|---|---|---|---|---|---|---|---|
| | | | WHO | | | wherein | | | |
| | | GenBank | IPD-KIR | | Base | the | | | |
| | | accession | accession | Closest | mutation | mutation | Codon | Amino acid | No. of |
| No. | Allele | number | number | allele | position | occurs | replacement | substitution | detection |
| 1 | 2DL1*00304 | KT438851 | IWS40002146 | 2DL1*00302 | CDS582 G>A | 5 | 173 GGG>GGA | no change | 1 |
| 2 | 2DL1*0030 | KT438852 | IWS4000214 | 2DL1*0030 | CDS1044 | 9 | 327 | no change | 1 |
| 3 | 2DL1*0030 | MZ361341 | IWS4000270 | 2DL1*0030 | CDS825 G>A | 8 | 254 | no change | 1 |
| 4 | 2DL1*0030 | MZ673561 | IWS4000271 | 2DL1*0030 | CDS102 C>T | 4 | 13 CAC>CAT | no change | 1 |
| 5 | 2DL1*0040 | MZ673558 | IWS4000271 | 2DL1*0040 | CDS144 A>G | 4 | 27 CAA>CAG | no change | 10 |
| 6 | 2DL1*030 | KP025959 | IWS4000198 | 2DL1*0030 | CDS343 A>G | 4 | 94 AGT>GGT | Ser>Gly | 1 |
| 7 | 2DL1*031 | KP025960 | IWS4000198 | 2DL1*0030 | CDS188 A>G | 4 | 42 | Glu>Gly | 1 |
| 8 | 2DL1*033 | KT438853 | IWS4000215 | 2DL1*0030 | CDS867 C>G | 8 | 268 | Ser>Arg | 1 |
| 9 | 2DL1*034 | KT438854 | IWS4000215 | 2DL1*0030 | CDS13 G>T | 1 | −17 GTC>TTC | Val>Phe | 1 |
| 10 | 2DL1*069 | MZ488844 | IWS40002702 | 2DL1*00302 | CDS986 T>C | 9 | 308 | Ile>Thr | 18 |
| | | | | | CDS989 T>G | 9 | 309 ATC>AGA | Ile>Arg | |
| | | | | | CDS990 C>A | 9 | | | |
| 11 | 2DL1*070 | MZ673559 | IWS4000271 | 2DL1*0040 | CDS110 G>C | 4 | 16 CGC>CCC | Arg>Pro | 3 |
| 12 | 2DL1*071 | MZ673560 | IWS4000271 | 2DL1*0030 | CDS668 T>C | 6 | 209 | Ser>Pro | 2 |
| 13 | 2DL1*072 | MZ673562 | IWS4000271 | 2DL1*0030 | CDS415 G>T | 5 | 118 | Val>Phe | 1 |
| 14 | 2DL1*073 | MZ673563 | IWS4000272 | 2DL1*0020 | CDS266 G>A | 4 | 68 CGC>CAC | Arg>His | 1 |
| 15 | 2DL1*074 | MZ673564 | IWS4000272 | 2DL1*0030 | CDS701 C>A | 6 | 213 | Pro>Gln | 1 |
| 16 | 2DL2*013 | KM017076 | IWS40001890 | 2DL2*00302 | CDS806 C>G | 7 | 248 | Ser>Cys | 1 |
| | | | | | CDS1018 | 9 | 319 | Ser>Pro | |
| 17 | 2DL3*0010 | KF766495 | IWS4000185 | 2DL3*0010 | CDS478 C>T | 5 | 139 | no change | 4 |
| 18 | 2DL3*0011 | KF766497 | IWS4000186 | 2DL3*0010 | CDS618 A>C | 5 | 185 | no change | 1 |
| 19 | 2DL3*0011 | MZ361338 | IWS4000269 | 2DL3*0010 | CDS564 C>G | 5 | 167 | no change | 3 |
| 20 | 2DL3*0011 | MZ361340 | IWS4000269 | 2DL3*0010 | CDS594 C>T | 5 | 177 | no change | 3 |
| 21 | 2DL3*025 | KF766496 | IWS4000186 | 2DL3*0010 | CDS280 C>A | 4 | 73 CTT>ATT | Leu>Ile | 1 |
| 22 | 2DL3*026 | KF766498 | IWS4000186 | 2DL3*0010 | CDS202 G>A | 4 | 47 GAC>AAC | Asp>Asn | 1 |
| 23 | 2DL3*027 | KF766499 | IWS4000186 | 2DL3*0010 | CDS809 G>C | 7 | 249 | Cys>Ser | 1 |
| 24 | 2DL3*028 | KF766500 | IWS4000187 | 2DL3*0010 | CDS800 G>A | 7 | 246 | Arg>His | 1 |
| 25 | 2DL3*029 | KF766501 | IWS4000187 | 2DL3*0010 | CDS505 C>A | 5 | 148 | Arg>Ser | 1 |
| 26 | 2DL3*030 | KF766502 | IWS4000187 | 2DL3*0010 | CDS201 G>C | 3 | 46 | Lys>Asn | 2 |
| 27 | 2DL3*031 | KF849247 | IWS40001877 | 2DL3*00101 | CDS735 T>C | 7 | 224 | no change | 1 |
| | | | | | CDS736 G>A | 7 | 225 | Val>Ile | |
| 28 | 2DL3*032 | KF849248 | IWS4000187 | 2DL3*0010 | CDS754 G>C | 5 | 231 | Val>Leu | 2 |
| 29 | 2DL3*037 | MZ361339 | IWS4000269 | 2DL3*0010 | CDS994 G>C | 9 | 311 | Val>Leu | 1 |
| 30 | 2DL4*0010 | KY974371 | IWS4000235 | 2DL4*0010 | CDS984 C>T | 9 | 305 | no change | 1 |
| 31 | 2DL4*0010 | MZ334488 | IWS4000269 | 2DL4*0010 | CDS624 C>T | 5 | 185 | no change | 2 |
| 32 | 2DL4*0050 | KT438855 | IWS4000215 | 2DL4*0050 | CDS888 G>A | 9 | 273 | no change | 2 |
| 33 | 2DL4*0050 | KT438856 | IWS4000215 | 2DL4*0050 | CDS987 G>T | 9 | 306 | no change | 2 |
| 34 | 2DL4*032 | KT438858 | IWS4000216 | 2DL4*0010 | CDS223 A>G | 3 | 52 AAC>GAC | Asn>Asp | 1 |
| 35 | 2DL4*033 | KT438859 | IWS4000216 | 2DL4*0010 | CDS1012 | 9 | 315 | Pro>Ser | 1 |
| 36 | 2DL4*034 | KT438857 | IWS4000215 | 2DL4*0050 | CDS200 G>T | 3 | 44 GGG>GTG | Gly>Val | 1 |
| 37 | 2DL4*039 | KY974369 | IWS4000234 | 2DL4*0060 | CDS412 G>A | 5 | 115 | Ala>Thr | 1 |
| 38 | 2DL4*040 | KY974370 | IWS4000234 | 2DL4*0010 | CDS524 A>G | 5 | 152 | Asn>Ser | 1 |
| 39 | 2DL5A*022 | KT438863 | IWS4000217 | 2DL5A*005 | CDS289 G>A | 3 | 76 GGT>AGT | Gly>Ser | 1 |
| 40 | 2DS2*009 | KT438862 | IWS4000216 | 2DS2*0010 | CDS109 C>T | 4 | 16 CCC>TCC | Pro>Ser | 3 |
| 41 | 2DS2*022 | MW65081 | IWS4000268 | 2DS2*0010 | CDS26 C>T | 1 | −13 | Ala>Val | 1 |
| 42 | 2DS4*0010 | KP025962 | IWS4000198 | 2DS4*0010 | CDS657 C>T | 5 | 198 | no change | 1 |
| 43 | 2DS4*017 | KP025961 | IWS4000198 | 2DS4*0010 | CDS707 C>G | 6 | 215 | Ser>Cys | 1 |
| 44 | 2DS4*018 | KP025963 | IWS4000198 | 2DS4*010 | CDS316 C>G | 4 | 85 CAC>GAC | His>Asp | 2 |
| 45 | 3DL1*0070 | MW56012 | IWS4000267 | 3DL1*0070 | CDS732 T>C | 5 | 223 | no change | 1 |
| 46 | 3DL1*0070 | MZ334486 | IWS4000269 | 3DL1*0070 | CDS162 G>A | 3 | 33 | no change | 1 |
| 47 | 3DL1*0150 | KF849249 | IWS4000188 | 3DL1*0150 | CDS906 C>T | 5 | 281 | no change | 1 |
| 48 | 3DL1*0150 | MZ334487 | IWS4000268 | 3DL1*0150 | CDS1008 | 7 | 315 | no change | 1 |
| 49 | 3DL1*0150 | MZ673556 | IWS4000270 | 3DL1*0150 | CDS519 T>C | 4 | 152 | no change | 1 |
| 50 | 3DL1*079 | KF849250 | IWS4000188 | 3DL1*0150 | CDS1119 | 8 | 352 | Met>Ile | 1 |
| 51 | 3DL1*120 | MZ673555 | IWS4000270 | 3DL1*0050 | CDS5 C>T | 1 | −20 | Ser>Leu | 1 |
| 52 | 3DL1*121 | MZ673557 | IWS4000270 | 3DL1*0010 | CDS337 G>C | 3 | 92 GTG>CTG | Val>Leu | 4 |
| 53 | 3DL2*0070 | KT899864 | IWS4000207 | 3DL2*0070 | CDS495 A>G | 4 | 144 | no change | 2 |
| 54 | 3DL2*0070 | KT899868 | IWS4000208 | 3DL2*0070 | CDS783 C>A | 5 | 240 | no change | 2 |
| 55 | 3DL2*083 | KT899867 | IWS4000208 | 3DL2*008 | CDS292 T>A | 3 | 77 TCA>ACA | Ser>Thr | 1 |
| 56 | 3DL2*084 | KT438861 | IWS4000216 | 3DL2*0090 | CDS502 G>A | 4 | 147 | Val>Ile | 1 |
| 57 | 3DL2*091 | KT438860 | IWS4000216 | 3DL2*0100 | CDS1315 | 9 | 418 | Lys>Gln | 3 |
| 58 | 3DL2*093 | KT899866 | IWS4000208 | 3DL2*0070 | CDS532 G>A | 4 | 157 | Ala>Thr | 6 |
| 59 | 3DL2*099 | KT899865 | IWS40002079 | 3DL2*00902 | CDS296 G>A | 3 | 77 CGC>CAC | Arg>His | 1 |
| | | | | | CDS308 T>C | 3 | 82 CTC>CCC | Leu>Pro | |
| 60 | 3DL3*0100 | KU529275 | IWS4000219 | 3DL3*0100 | CDS408 G>A | 4 | 115 | no change | 1 |
| 61 | 3DL3*0260 | KU529271 | IWS4000218 | 3DL3*0260 | CDS1074 | 8 | 337 | no change | 5 |
| 62 | 3DL3*0480 | KU529269 | IWS4000217 | 3DL3*0480 | CDS1074 | 8 | 337 | no change | 12 |
| 63 | 3DL3*062 | KU529272 | IWS40002186 | 3DL3*02601 | CDS1074 | 8 | 337 | no change | 3 |
| | | | | | CDS1184 | 9 | 374 | Thr>Ile | |
| 64 | 3DL3*063 | KU529270 | IWS40002182 | 3DL3*04801 | CDS1074 | 8 | 337 | no change | 2 |
| | | | | | CDS1184 | 9 | 374 | Thr>Ile | |
| 65 | 3DL3*064 | KU529273 | IWS4000218 | 3DL3*0060 | CDS1184 | 9 | 374 | Thr>Ile | 3 |
| 66 | 3DL3*065 | KU529274 | IWS4000219 | 3DL3*0010 | CDS1184 | 9 | 374 | Thr>Ile | 2 |
| 67 | 3DS1*078 | KJ001806 | IWS4000190 | 3DS1*0130 | CDS775 G>C | 5 | 238 | Gly>Arg | 1 |
| 68 | 3DS1*082 | KJ001804 | IWS4000190 | 3DS1*0130 | CDS1114 | 8 | 351 | no change | 2 |
| 69 | 3DS1*083 | KJ001805 | IWS40001907 | 3DS1*01301 | CDS393 T>G | 4 | 110 | no change | 1 |
| | | | | | CDS400 G>C | 4 | 113 | Val>Leu | |
| | | | | | CDS416 G>C | 4 | 118 | Arg>Thr | |
| 70 | 3DS1*084 | KJ001807 | IWS4000191 | 3DS1*0130 | CDS308 C>T | 3 | 82 CCC>CTC | Pro>Leu | 1 |
| 71 | 3DS1*085 | KJ365317 | IWS4000191 | 3DS1*0130 | CDS934 C>T | 5 | 291 CTT>TTT | Leu>Phe | 2 |
| 72 | 3DL3*0150 | OP204419 | IWS4000276 | 3DL3*0150 | CDS969 C>T | 7 | 302 | no change | 12 |
| 73 | 3DL3*116 | OP204420 | IWS4000276 | 3DL3*0090 | CDS724 T>G | 5 | 221 | Leu>Val | 6 |
| 74 | 3DL3*117 | OP204421 | IWS40002764 | 3DL3*01002 | CDS336 T>C | 3 | 91 CCT>CCC | no change | 3 |
| | | | | | CDS337 G>C | 3 | 92 GTG>CTG | Val>Leu | |
| 75 | 3DL3*118 | OP204422 | IWS4000276 | 3DL3*0100 | CDS502 A>G | 4 | 147 | Ile>Val | 14 |

The detection reagent for PCR amplification of the full-length sequences of the 16 KIR genes included: specific PCR primer pairs, DNA polymerase mixture for rapid amplification of long-fragment, genomic DNA and ddH₂O.

In this example, a sample was randomly selected, which was previously subjected to detection of the presence or absence of KIR genes at the low-resolution level by Q-PCR, with the result indicating a KIR AA1 genotype (carrying a total of 9 KIR genes: 2DL1, 2DL3, 2DL4, 2DS4, 3DL1, 3DL2, 3DL3, 2DP1 and 3DP1). Third-generation long-read sequencing-based typing was performed on all KIR genes carried by the sample using the present disclosure to verify the implementation effect of the present disclosure.
i) In the present disclosure, the full-length sequences of all KIR genes carried by the sample were first amplified using 3 pairs of KIR gene-specific primers. The amplification reaction was performed on an ABI Veriti PCR instrument. The compositions of the PCR amplification reaction systems were respectively as follows:

The first PCR primer pair was used for specific multiplex PCR amplification of 14 KIR genes excluding 2DL4 and 3DP1, and the second PCR primer pair was used for amplification of the 2DL4 gene. The compositions of the PCR amplification reaction systems thereof were both as follows:

| | |
|---|---|
| DNA polymerase mixture for rapid amplification of | 12.5 μL; |
| long-fragment | |
| 10 μM PCR primers | 1.0 μL each; |
| 50-100 ng/μL genomic DNA | 2.0 μL; and |
| ddH2O to make up to | 25.0 μL. |

The third PCR primer pair was used for specific amplification of the full-length sequence of the 3DP1 gene and a partial sequence of the first KIR gene in an upstream flanking region thereof. The composition of the PCR amplification reaction system thereof consisted of:

| | |
|---|---|
| DNA polymerase mixture for rapid amplification of | 12.5 μL; |
| long-fragment | |
| 1.0 μM PCR primers | 3.0 μL each; |
| 50-100 ng/μL genomic DNA | 2.0 μL; and |
| ddH2O to make up to | 25.0 μL. |

The PCR amplification conditions were respectively as follows:

The first PCR primer pair was used for specific amplification of 14 KIR genes excluding 2DL4 and 3DP1, and the amplification conditions therefor were as follows: 98° C. for 2 min; 98° C. for 10 Sec; 59° C. for 10 Sec; 72° C. for 4 min, for 35 cycles; 72° C. for 5 min; and 4° C. for Infinite.

The second PCR primer pair was used for specific amplification of the 2DL4 gene, and the amplification conditions therefor were as follows: 98° C. for 2 min; 98° C. for 10 Sec; 56° C. for 10 Sec; 72° C. for 4 min, for 35 cycles; 72° C. for 5 min; and 4° C. for Infinite.

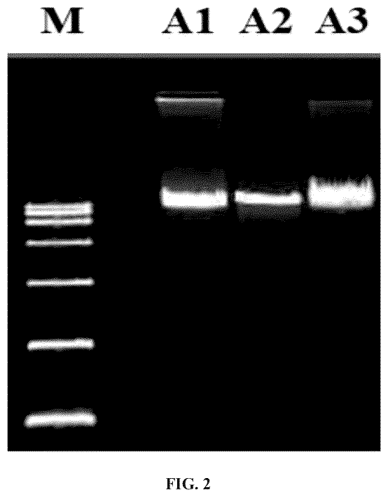

The third PCR primer pair was used for specific amplification of the 3DP1 gene and a partial sequence of the first KIR gene in an upstream flanking region thereof, and the amplification conditions therefor were as follows: 98° C. for 2 min; 98° C. for 10 Sec; 65° C. for 10 Sec; 72° C. for 5 min, for 35 cycles; 72° C. for 5 min; and 4° C. for Infinite.
ii) Electrophoresis of PCR amplification products: 10.0 μL of PCR products were taken, stained with a nucleic acid dye, and subjected to electrophoresis on a 0.8% agarose gel. With reference to the Takara DL15000 DNA marker, the bands of specific PCR amplification products were observed in a gel imaging system. Clear and bright target bands were observed in the products amplified by the first, second and third pairs of PCR primers. The electrophoresis image of the PCR products is shown in detail in FIG. 2. In FIG. 2, M: DL15000 Marker; A1: Amplicon 1, the full-length sequences of 14 KIR genes (excluding 2DL4 and 3DP1) specifically amplified by the first PCR primer pair via multiplex PCR; A2: Amplicon 2, the full-length sequence of the 2DL4 gene specifically amplified by the second PCR primer pair; A3: the full-length sequence of the 3DP1 gene and a partial sequence of the first KIR gene in an upstream flanking region thereof, specifically amplified by the third PCR primer pair.
iii) Quality inspection of PCR amplification products: 1.0 μL of the products amplified by the first, second and third pairs of PCR primers were respectively taken. The purity was detected by Nanodrop, and the concentration was detected using a Qubit 4.0 instrument.
iv) Mixing and purification of amplification products: The products amplified by the first, second and third pairs of PCR primers were mixed into one tube at a certain mass ratio (30:2:3). After mixing, 1.0 μg of the amplification products was taken, and the total volume was made up to 100 μL with deionized water. Purification was performed twice by the AMpure magnetic bead method, and the concentration of the purified amplification products was measured using a Qubit 4.0 instrument.
v) Library construction: 300-500 ng of the amplification products were taken, and the library was constructed using the PacBio SMRTEBLL® PREP KIT 3.0 kit.

The amplification products were first subjected to end repair and A-base addition. The reaction system contained 8.0 μL of Repair buffer, 4.0 μL of End Repair Mix, 2.0 μL of DNA Repair mix, 40.0 μL of the products, and 6.0 μL of RNase-free water, totaling 60.0 μL of the reaction mixture. Incubation was performed at 37° C. for 30 minutes and at 65° C. for 5 minutes to allow the target amplification products to form sticky ends with dA tails for ligation with adapters carrying T tails.

Adapter ligation: The reaction system contained 4.0 μL of SMRTbell adapter, 30.0 μL of Ligation mix, 1.0 μL of Ligation enhancer, and 60.0 μL of the products subjected to end repair and A-base addition, totaling 95.0 μL of the reaction mixture. Incubation was performed at 20° C. for 30 minutes. After adding 95.0 μL of SMRTbell cleanup beads to the adapter-ligated products for purification, 40.0 μL of buffer was added for elution.

Third step: Nuclease treatment: The reaction system contained 5.0 μL of Nuclease buffer, 5.0 μL of Nuclease mix, and 40.0 μL of the purified adapter-ligated products, totaling 50.0 μL of the reaction mixture. Incubation was performed at 37° C. for 15 minutes. After adding 50.0 μl of AMpure beads to the digestion products for purification, 15.0 μL of buffer was added for elution.

Finally, library quality inspection was performed, and the concentration of the library was detected using a Qubit instrument.
vi) Library preparation before on-machine sequencing: The library of the sample in this example was mixed with the libraries of other samples at an equal ng amount. AMpure magnetic beads with a volume of 0.6 times the volume of the mixed library sample were added for purification, and all target large fragments were recovered.
vii) On-machine library preparation: About 70 ng of the mixed amplicon library was taken. The on-machine library was prepared using the SEQUEL® II BINDING KIT 3.2 kit. Sequencing primers from the kit were added, and incubation was performed at 20° C. for 30 min to allow the sequencing primers to bind to the adapter sequence positions of the library. Subsequently, sequencing enzyme from the kit was added, and incubation was performed at 30° C. for 30 min to immobilize the sequencing enzyme on the sequencing primers. The prepared product was purified using the magnetic beads from the kit to obtain the on-machine library.
viii) On-machine sequencing: 100 μmol/L of the prepared product for on-machine sequencing was placed on a PacBio Sequel II high-throughput gene sequencer for sequencing. The on-machine sequencing reaction was completed using the SEQUEL II SEQUENCING KIT 2.0 and PacBio SMRT Cell 8M chip.
ix) Bioinformatics analysis and KIR genotyping:

First step: Data pre-processing: After sequencing was completed, the system (SMRT Link, v13.0, Pacific Bioscience) configured with the PacBio Sequel II sequencer automatically converted the fluorescence signals into base sequence information. Simultaneously, intra-molecule sequence correction and subsequent filtering were performed to obtain HIFI reads with a single-molecule accuracy above Q20, thereby forming BAM files.

Second step: Data demultiplexing: The official built-in PacBio software Lima was used to demultiplex the HIFI reads for distinguishing different samples. The number of bases, mean read length, and sequence quality of HIFI reads for Sample No. 13013519 in this example are shown in detail in Table 7.

TABLE 7
Number of bases, mean read length, and sequence quality
of HIFI reads for Sample No. 13013519 in Example 1

| Sample | Read | Base | | |
|---|---|---|---|---|
| Number | Number | Number | Mean Length | Mean Read Quality |
| 13013519 | 56135 | 471968267 | 8407 | 0.999 |

Third step: Data alignment: The official built-in PacBio software pbMM2 was used for alignment against the hg38 reference genome. Software such as samtools and bedtools was used to extract gene sequences from the target chromosomes, thereby generating standard fasta files. Sample No. 13013519 in this example is the KIR AA1 genotype. The read lengths and number of reads of the 9 KIR genes carried thereby are shown in Table 8.

TABLE 8
Statistics of the off-machine data for
Sample No. 13013519 in Example 1

| No. | KIR gene | Length (kb) | Number of reads |
|---|---|---|---|
| 1 | 3DL3 | 12.0 | 1708 |
| 2 | 2DL3 | 14.4 | 1766 |
| 3 | 2DP1 | 12.7 | 6301 |
| 4 | 2DL1 | 14.4 | 1628 |
| 5 | 3DP1 | 16.4 | 2433 |
| 6 | 2DL4 | 11.1 | 5548 |
| 7 | 3DL1 | 14.2 | 5664 |
| 8 | 2DS4 | 15.7 | 1405 |
| 9 | 3DL2 | 16.6 | 2426 |

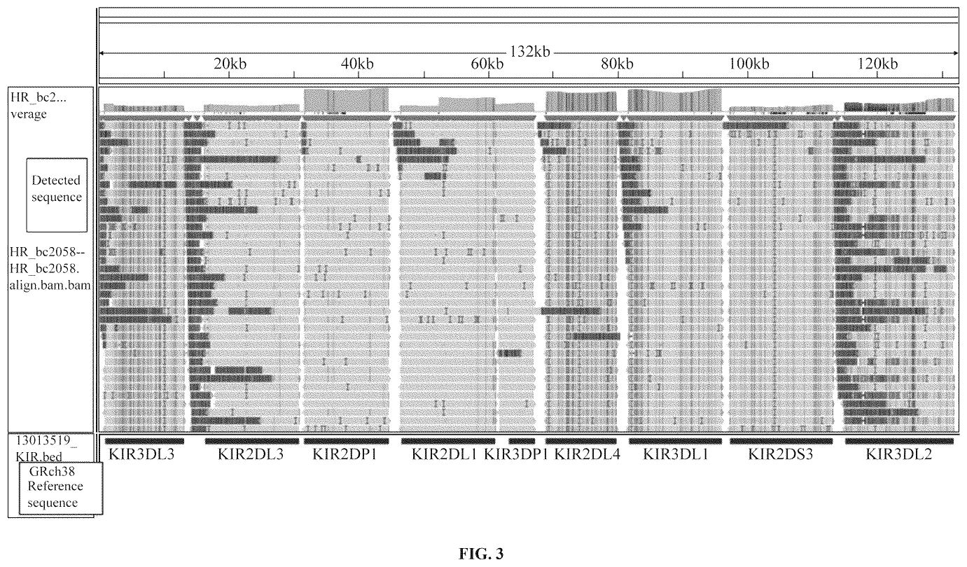

Fourth step: Data filtering: Using the same method as described above, the above fasta sequence data were aligned to the reference sequence of each KIR gene, and sequences of non-target genes were filtered out. The alignment results are shown in detail in FIG. 3. As can be seen from the visualization diagram of FIG. 3, the target sequences of all 9 KIR genes (2DL1, 2DL3, 2DL4, 2DS4, 3DL1, 3DL2, 3DL3, 2DP1 and 3DP1) carried by the sample in Example 1 can be clearly aligned to the reference genome.

Fifth step: Coding sequence (CDS) extraction: The CDS sequences of KIR genes were extracted using third-party software such as sam2tsv.

Sixth step: Typing: The extracted CDS sequences were aligned to the CDS sequences of all KIR alleles in the IPD-KIR database. If the obtained CDS sequences showed 100% consistency with a certain genotyping result in the database and reached a certain depth, they were classified into that genotype.

The KIR genotype of Sample No. 13013519 in Example 1 can be determined as: 3DL3*01001,01002-2DL3*00101-2DP1*00201-2DL1*00302-3DP1*00302-2DL4*00102-3DL1 *01502-2DS4*00101-3DL2*00201.

### REFERENCES

[21] Deng Z, Zhen J, Harrison G F, et al. Adaptive Admixture of HLA Class I Allotypes Enhanced Genetically Determined Strength of Natural Killer Cells in East Asians. Mol Biol Evol. 2021, 38(6): 2582-2596.
[22] Deng Z, Zhen J, Zhu B, Zhang G, Yu Q, Wang D, Xu Y, He L, Lu L. Allelic Diversity of KIR3DL1/3DS1 in a Southern Chinese Population. Hum Immunol. 2015, 76(9): 663-666.
[23] Zhen J, He L, Xu Y, et al. Allelic Polymorphism of KIR2DL2/2DL3 in a Southern Chinese Population. Tissue Antigens. 2015, 86(5): 362-367.

### Example 2

In this example, a sample was randomly selected, which was previously subjected to detection of the presence or absence of KIR genes at the low-resolution level by Q-PCR, with the result indicating a KIR AB8 genotype (carrying a total of 13 KIR genes: 2DL1, 2DL3, 2DL4, 2DL5, 2DS1, 2DS3, 2DS4, 3DL1, 3DL2, 3DL3, 3DS1, 2DP1 and 3DP1). Third-generation long-read sequencing-based typing was performed on all KIR genes carried by the sample using the present disclosure to verify the implementation effect of the present disclosure.

The experimental methods of Example 2 (including PCR amplification, electrophoresis and quality inspection of the amplification products, library construction, on-machine library preparation, on-machine sequencing, bioinformatics analysis and KIR genotyping) were consistent with the experimental methods of Example 1.

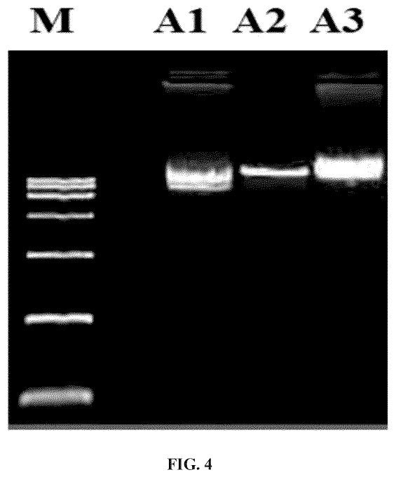

The electrophoresis image of the PCR products of Example 2 is shown in detail in FIG. 4. In FIG. 4, M: DL15000 Marker; A1: Amplicon 1, the full-length sequences of 14 KIR genes (excluding 2DL4 and 3DP1) specifically amplified by the first PCR primer pair via multiplex PCR; A2: Amplicon 2, the full-length sequence of the 2DL4 gene specifically amplified by the second PCR primer pair; A3: the full-length sequence of the 3DP1 gene and a partial sequence of the first KIR gene in an upstream flanking region thereof, specifically amplified by the third PCR primer pair.

In the bioinformatics analysis, the number of bases, mean read length, and sequence quality of HIFI reads for Sample No. 13013484 in Example 2 are shown in detail in Table 9.

TABLE 9
Number of bases, mean read length, and sequence quality
of HIFI reads for Sample No. 13013484 in Example 2

| Sample | Reads | Base | | |
|---|---|---|---|---|
| Number | Number | Number | Mean Length | Mean Read Quality |
| 13013484 | 27789 | 271152437 | 9757 | 0.9991 |

In the bioinformatics analysis, sample No. 13013484 in Example 2 is the KIR AB8 genotype. The read lengths and number of reads of the 13 KIR genes carried thereby are as shown in Table 10.

TABLE 10
Statistics of the off-machine data for
Sample No. 13013484 in Example 2

| No. | KIR gene | Length (kb) | Number of reads |
|---|---|---|---|
| 1 | 3DL3 | 12.0 | 352 |
| 2 | 2DL3 | 14.4 | 202 |
| 3 | 2DP1 | 12.7 | 1443 |
| 4 | 2DL1 | 14.4 | 336 |
| 5 | 3DP1 | 16.4 | 880 |
| 6 | 2DL4 | 11.1 | 1871 |
| 7 | 3DL1 | 14.2 | 1238 |
| 8 | 2DS4 | 15.7 | 659 |
| 9 | 3DL2 | 16.6 | 507 |
| 10 | 2DL5 | 9.3 | 11902 |
| 11 | 2DS1 | 14.4 | 1223 |
| 12 | 2DS3 | 14.7 | 234 |
| 13 | 3DS1 | 14.6 | 343 |

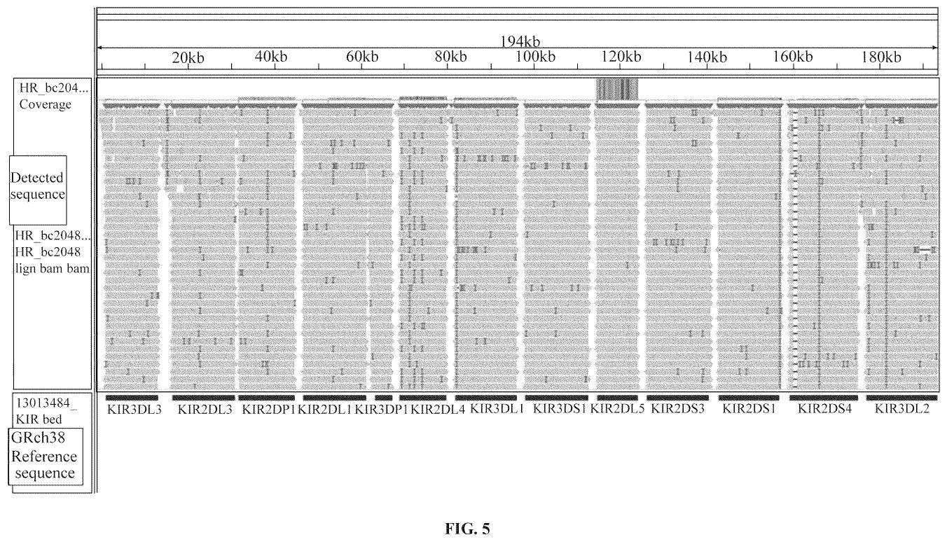

The fasta sequence data of Example 2 were aligned to the reference sequence of each specific KIR gene, and sequences of non-target genes were filtered out. The obtained alignment results are shown in detail in FIG. 5. As can be seen from the visualization diagram of FIG. 5, the target sequences of all 13 KIR genes (2DL1, 2DL3, 2DL4, 2DL5, 2DS1, 2DS3, 2DS4, 3DL1, 3DL2, 3DL3, 3DS1, 2DP1 and 3DP1) carried by the sample in Example 2 can be clearly aligned to the reference genome.

Based on the alignment results between the extracted CDS sequences and the CDS sequences of all KIR alleles in the IPD-KIR database, the KIR genotype of Sample No. 13013484 in Example 2 can be determined as:
3DL3*00802,01002-2DL3*00101-2DP1*00201-2DL1*00302,06901-3DP1*00302-2DL4*0010 3,00501-3DL1*02001-3DS1*085-2DL5A*00501-2DS3*00201-2DS1*00201-2DS4*00101-3DL 2*00201,00701.

### Example 3

In this example, the genotyping of KIR genes based on the third-generation long-read sequencing was performed on samples from 20 unrelated healthy individuals using the present disclosure. These samples had previously been subjected to KIR genotyping after KIR-related sequences had been extracted following whole-genome sequencing (hereinafter referred to as “KIR genotyping by whole-genome sequencing”). The results of sequencing-based typing of KIR amplicons in the present disclosure were compared with the results of “KIR genotyping by whole-genome sequencing” to verify the implementation effect of the present disclosure.

The experimental methods of Example 3 (including PCR amplification, electrophoresis and quality inspection of the amplification products, library construction, on-machine library preparation, on-machine sequencing, bioinformatics analysis and KIR genotyping) were consistent with the experimental methods of Example 1. The difference was that the test samples in Example 3 were samples from 20 unrelated healthy individuals.

The KIR genotypes of the 20 samples in Example 3 are shown in detail in Table 11. The results of sequencing-based typing of KIR amplicons in the present disclosure were consistent with the results of “KIR genotyping by whole-genome sequencing”.

TABLE Il
Results of third-generation long-rend sequencing-based typing of 16 KIR genes in the population (n = 20)

| No. | | 2DL1 | 2DL2 | 2DL3 | 2DL4 | 2DL5 | 2DS1 | 2DS2 | 2DS3 | 2DS4 | 2DS5 | 3DL1 | 3DS1 | 3DL2 | 3DL3 | 2DP1 | 3DP1 |
|---|---|---|---|---|---|---|---|---|---|---|---|---|---|---|---|---|---|
| 1 | Results of | 00201, | | 00101, | 00102 | | | | | 00101 | | 01502 | | 002 | 010, | 00301, | 00601, |
| | the present | 00302 | | 00201 | | | | | | | | | | | 02602 | 00201 | 00302 |
| | disclosure | | | | | | | | | | | | | | | | |
| | Genomic | 00201, | | 00101, | 00102 | | | | | 00101 | | 01502 | | 002 | 010, | 00301, | 00601, |
| | sequencing | 00302 | | 00201 | | | | | | | | | | | 02602 | 00201 | 00302 |
| | results | | | | | | | | | | | | | | | | |
| 2 | Results of | 00302 | | 00101, | 00102, | | | | | 00101, | | 01502, | | 002 | 009, | 00201 | 00302 |
| | the present | | | 019 | 011 | | | | | 010 | | 00501 | | | 010 | | |
| | disclosure | | | | | | | | | | | | | | | | |
| | Genomic | 00302 | | 00101, | 00102, | | | | | 00401, | | 01502, | | 002 | 009, | 00201 | 00302 |
| | sequencing | | | 019 | 011 | | | | | 010 | | 00501 | | | 010 | | |
| | results | | | | | | | | | | | | | | | | |
| 3 | Results of | 00201, | | 00101, | 00602, | | | | | 00401, | | 00501, | | 008, | 00901, | 00201, | 00302, |
| | the present | 00302 | | 00201 | 011 | | | | | 010 | | 00701 | | 010 | 02602 | 00301 | 00601 |
| | disclosure | | | | | | | | | | | | | | | | |
| | Genomic | 00201, | | 00101, | 00602, | | | | | 00401, | | 00501, | | 008, | 00901, | 00201, | 00302, |
| | sequencing | 00302 | | 00201 | 011 | | | | | 010 | | 00701 | | 010 | 02602 | 00301 | 00601 |
| | results | | | | | | | | | | | | | | | | |
| 4 | Results of | 00201 | | 00201 | 00501, | A*012 | 00201 | | | 010 | 00201 | 00501 | 084 | 002, | 001, | 00301 | 01501. |
| | the present | | | | 011 | | | | | | | | | 00701 | 04802 | | 00601 |
| | disclosure | | | | | | | | | | | | | | | | |
| | Genomic | 00201 | | 00201 | 00501, | A*012 | 00201 | | | 010 | 00201 | 00501 | 084 | 002, | 001, | 00301 | 01501. |
| | sequencing | | | | 011 | | | | | | | | | 00701 | 04802 | | 00601 |
| | results | | | | | | | | | | | | | | | | |
| 5 | Results of | 00302 | | 00101 | 00501, | A*001 | 00201 | | | 00301 | 00201 | 00101 | 01301 | 001, 093 | 008, | 019, | 00302, |
| | the present | | | | 00801 | | | | | | | | | | 010 | 00201 | 01001 |
| | disclosure | | | | | | | | | | | | | | | | |
| | Genomic | 00302 | | 00101 | 00501, | A*001 | 00201 | | | 00301 | 00201 | 00101 | 01301 | 001, 093 | 008, | 019, | 00302, |
| | sequencing | | | | 00801 | | | | | | | | | | 010 | 00201 | 01001 |
| | results | | | | | | | | | | | | | | | | |
| 6 | Results of | 00302 | 00301 | 00101 | 00102, | A*001 | 00201 | 00101 | | 00101 | 00201 | 01502 | 01301 | 002, | 008, | 00201 | 00302, |
| | the present | | | | 00501 | B*006 | | | | | | | | 00701 | 010 | | 007 |
| | disclosure | | | | | | | | | | | | | | | | |
| | Genomic | 00302 | 00301 | 00101 | 00102, | A*001 | 00201 | 00101 | | 00101 | 00201 | 01502 | 01301 | 002, | 008, | 00201 | 00302, |
| | sequencing | | | | 00501 | B*006 | | | | | | | | 00701 | 010 | | 007 |
| | results | | | | | | | | | | | | | | | | |
| 7 | Results of | 00302 | 00301, | 00101 | 00102, | | | 00101 | | 00101, | | 01502, | | 002, | 008, 010 | 00201 | 00302, |
| | the present | | 013 | | 006 | | | | | 00401 | | 00701 | | 021 | | | 00901 |
| | disclosure | | | | | | | | | | | | | | | | |
| | Genomic | 00302 | 00301, | 00101 | 00102, | | | 00101 | | 00101, | | 01502, | | 002, | 008, 010 | 00201 | 00302, |
| | sequencing | | 013 | | 006 | | | | | 00401 | | 00701 | | 021 | | | 00901 |
| | results | | | | | | | | | | | | | | | | |
| 8 | Results of | 00302 | | 00101 | 00102 | | | | | 00101 | | 01502 | | 00201 | 01001, | 00201 | 00302 |
| | the present | | | | | | | | | | | | | | 01002 | | |
| | disclosure | | | | | | | | | | | | | | | | |
| | Genomic | 00302 | | 00101 | 00102 | | | | | 00101 | | 01502 | | 00201 | 01001, | 00201 | 00302 |
| | sequencing | | | | | | | | | | | | | | 01002 | | |
| | results | | | | | | | | | | | | | | | | |
| 9 | Results of | 00302 | | 00101 | 00103, | A*005 | 00201 | | 00201 | 00101 | | 02001 | 085 | 00201, | 00802, | 00201 | 00302 |
| | the present | | | | 00501 | | | | | | | | | 00701 | 01002 | | |
| | disclosure | | | | | | | | | | | | | | | | |
| | Genomic | 00302 | | 00101 | 00103, | A*005 | 00201 | | 00201 | 00101 | | 02001 | 085 | 00201, | 00802, | 00201 | 00302 |
| | sequencing | | | | 00501 | | | | | | | | | 00701 | 01002 | | |
| | results | | | | | | | | | | | | | | | | |
| 10 | Results of | 00302 | 00301 | 00101 | 00501 | A*001 | 00201 | 00101 | | | 00201 | | 01301 | 00701, | 01001, | 00201 | 00302 |
| | the present | | | | | B*006 | | | | | | | | 015 | 02801 | | |
| | disclosure | | | | | | | | | | | | | | | | |
| | Genomic | 00302 | 00301 | 00101 | 00501 | A*001 | 00201 | 00101 | | | 00201 | | 01301 | 00701, | 01001, | 00201 | 00302 |
| | sequencing | | | | | B*006 | | | | | | | | 015 | 02801 | | |
| | results | | | | | | | | | | | | | | | | |
| 11 | Results of | 00302, | 00301 | 023 | 00102, | B*010 | | 00101 | 001 | 00101 | | 01502, | | 002, | 010 | 00201, | 00302 |
| | the present | 00401 | | | 00103 | | | | | | | 020 | | 009 | | 0030 | |
| | disclosure | | | | | | | | | | | | | | | | |
| | Genomic | 00302, | 00301 | 023 | 00102, | B*010 | | 00101 | 001 | 00101 | | 01502, | | 002, | 010 | 00201, | 00302 |
| | sequencing | 00401 | | | 00103 | | | | | | | 020 | | 009 | | 0030 | |
| | results | | | | | | | | | | | | | | | | |
| 12 | Results of | 00302, | 00101 | 00101 | 00102, | A*001 | 00201 | 00101 | 001 | 00101 | | 01502 | 01301 | 002, | 010, | 00201 | 00302, |
| | the present | 00401 | | | 00501 | B*002 | | | | | | | | 015 | 028 | | 00601 |
| | disclosure | | | | | | | | | | | | | | | | |
| | Genomic | 00302, | 00101 | 00101 | 00102, | A*001 | 00201 | 00101 | 001 | 00101 | | 01502 | 01301 | 002, | 010, | 00201 | 00302, |
| | sequencing | 00401 | | | 00501 | B*002 | | | | | | | | 015 | 028 | | 00601 |
| | results | | | | | | | | | | | | | | | | |
| 13 | Results of | 00302 | 00301 | 00101 | 00103, | A*005, | 00201 | 00101 | 00201 | 00101 | | 020 | 01301 | 00701, | 008, | 00201, | 00302 |
| | the present | | | | 00301 | B*010 | | | | | | | | 0099 | 010 | 00301 | |
| | disclosure | | | | | | | | | | | | | | | | |
| | Genomic | 00302 | 00301 | 00101 | 00103, | A*005 | 00201 | 00101 | 00201 | 00101 | | 020 | 01301 | 00701, | 008, | 00201, | 00302 |
| | sequencing | | | | 00301 | B*010 | | | | | | | | 0099 | 010 | 00301 | |
| | results | | | | | | | | | | | | | | | | |
| 14 | Results of | 00302 | | 00101 | 00501, | A*005 | 00201 | | 00201 | 00101, | | 00101 | 01301 | 001, | 009, | 00201 | 00302, |
| | the present | | | | 00801 | | | | | 010 | | | | 00701 | 010 | | 01001 |
| | disclosure | | | | | | | | | | | | | | | | |
| | Genomic | 00302 | | 00101 | 00501, | A*005 | 00201 | | 00201 | 00101, | | 00101 | 01301 | 001, | 009, | 00201 | 00302, |
| | sequencing | | | | 00801 | | | | | 010 | | | | 00701 | 010 | | 01001 |
| | results | | | | | | | | | | | | | | | | |
| 15 | Results of | 00302 | 00301 | 00101 | 00102 | B*006 | 00201 | 00101 | | 00101 | | 01502 | | 00701, | 008, | 00301 | 01501, |
| | the present | | | | | | | | | | | | | 039 | 010 | | 00601 |
| | disclosure | | | | | | | | | | | | | | | | |
| | Genomic | 00302 | 00301 | 00101 | 00102 | B*006 | 00201 | 00101 | | 00101 | | 01502 | | 00701, | 008, | 00301 | 01501, |
| | sequencing | | | | | | | | | | | | | 039 | 010 | | 00601 |
| | results | | | | | | | | | | | | | | | | |
| 16 | Results of | 00302 | 00101 | 00101 | 00102 | B*002 | 00201 | 00101 | 00201 | 00101 | | 01502 | | 002, | 010 | 00201 | 00302, |
| | the present | | | | | | | | | | | | | 00701 | | | 007 |
| | disclosure | | | | | | | | | | | | | | | | |
| | Genomic | 00302 | 00101 | 00101 | 00102 | B*002 | 00201 | 00101 | 00201 | 00101 | | 01502 | | 002, | 010 | 00201 | 00302, |
| | sequencing | | | | | | | | | | | | | 00701 | | | 007 |
| | results | | | | | | | | | | | | | | | | |
| 17 | Results of | 00302, | 00101 | 00101 | 00102, | A*005 | | 00101 | 001 | 00101 | | 01502 | 01301, | 002 | 009 | 00201 | 00302 |
| | the present | 00401 | | | 00501 | B*002 | | | | | | | 082 | | | | |
| | disclosure | | | | | | | | | | | | | | | | |
| | Genomic | 00302, | 00101 | 00101 | 00102, | A*005 | | 00101 | 001 | 00101 | | 01502 | 01301, | 002 | 009 | 00201 | 00302 |
| | sequencing | 00401 | | | 00501 | B*002 | | | | | | | 082 | | | | |
| | results | | | | | | | | | | | | | | | | |
| 18 | Results of | 00302 | | 00101 | 00102, | | | | | 00101 | | 01502 | 01301 | 002 | 010 | 00201 | 00302, |
| | the present | | | | 00501 | | | | | | | | | | | | 00601 |
| | disclosure | | | | | | | | | | | | | | | | |
| | Genomic | 00302 | | 00101 | 00102, | | | | | 00101 | | 01502 | 01301 | 002 | 010 | 00201 | 00302, |
| | sequencing | | | | 00501 | | | | | | | | | | | | 00601 |
| | results | | | | | | | | | | | | | | | | |
| 19 | Results of | 00302 | 00301 | 00101 | 00503 | A*001, | 00201 | 00101 | | | 00201 | | 01301 | 00701 | 008, | 00201 | 00302, |
| | the present | | | | | A*012 | | | | | | | | | 010 | | 00901 |
| | disclosure | | | | | | | | | | | | | | | | |
| | Genomic | 00302 | 00301 | 00101 | 00503 | A*001, | 00201 | 00101 | | | 00201 | | 01301 | 00701 | 008, | 00201 | 00302, |
| | sequencing | | | | | A*012 | | | | | | | | | 010 | | 00901 |
| | results | | | | | | | | | | | | | | | | |
| 20 | Results of | 00302 | 00301 | 00101 | 00501 | A*001, | 00201 | 00101 | 00201 | | 00201 | | 01301 | 00701 | 008, | 00201 | 00302 |
| | the present | | | | | B*010 | | | | | | | | | 010 | | |
| | disclosure | | | | | | | | | | | | | | | | |
| | Genomic | 00302 | 00301 | 00101 | 00501 | A*001, | 00201 | 00101 | 00201 | | 00201 | | 01301 | 00701 | 008, | 00201 | 00302 |
| | sequencing | | | | | B*010 | | | | | | | | | 010 | | |
| | results | | | | | | | | | | | | | | | | |

What is claimed is:

1. A PCR amplification primer set for amplifying full-length sequences of 16 KIR genes, comprising 3 pairs of specific PCR primers, wherein the 3 pairs of specific PCR primers comprise a first PCR primer pair, a second PCR primer pair, and a third PCR primer pair;
sequences of the first PCR primer pair are set forth in SEQ ID NOs. 1-2, sequences of the second PCR primer pair are set forth in SEQ ID NOs. 3-4, and sequences of the third PCR primer pair are set forth in SEQ ID NOs. 5-6;
the first PCR primer pair is used for specific multiplex PCR amplification of the full-length sequences of the 2DL1, 2DL2, 2DL3, 2DL5, 2DS1, 2DS2, 2DS3, 2DS4, 2DS5, 3DL1, 3DL2, 3DL3, 3DS1 and 2DP1 genes, the second PCR primer pair is used for specific amplification of the full-length sequence of the 2DL4 gene, and the third PCR primer pair is used for specific amplification of the full-length sequence of the 3DP1 gene and a partial sequence of one KIR gene closest to the 3DP1 gene in an upstream flanking region thereof.

2. The PCR amplification primer set for amplifying full-length sequences of 16 KIR genes according to claim 1, wherein in the third PCR primer pair used for specific amplification of the full-length sequence of the 3DP1 gene and a partial sequence of one KIR gene closest to the 3DP1 gene in an upstream flanking region thereof, the one KIR gene closest to the 3DP1 gene in the upstream flanking region thereof comprises any one of the 2DL1, 2DL2, 2DL3, 2DL5, 2DS2, 2DS3, 2DS5, 3DL3 and 2DP1 genes; and
the partial sequence of the one KIR gene closest to the 3DP1 gene comprises a sequence from intron 5 to a 3′-untranslated region of the KIR gene closest to the 3DP1 gene.

3. The PCR amplification primer set for amplifying full-length sequences of 16 KIR genes according to claim 1, wherein the PCR amplification primer set is used for amplification of the full-length sequences of all 16 KIR genes in humans by long-range PCR and for library construction in third-generation long-read sequencing-based typing of KIR genes;
lengths of amplification products of the first PCR primer pair, the second PCR primer pair and the third PCR primer pair range from 9.2 kb to 16.7 kb, covering all exons and introns of each KIR gene.

4. A PCR amplification kit for full-length sequences of 16 KIR genes, comprising the PCR amplification primer set for full-length sequences of 16 KIR genes as claimed in claim 1, and a PCR amplification detection reagent.

5. A method for genotyping 16 KIR genes based on third-generation long-read sequencing, comprising steps of:
1) mixing a first PCR primer pair, a second PCR primer pair and a third PCR primer pair separately with genomic DNA of a test sample, DNA polymerase mixture for rapid amplification of long fragment and ddH₂O to prepare PCR amplification reaction systems, thereby obtaining a first PCR amplification reaction system, a second PCR amplification reaction system and a third PCR amplification reaction system, wherein sequences of the first PCR primer pair are set forth in SEQ ID NOs. 1-2, sequences of the second PCR primer pair are set forth in SEQ ID NOs. 3-4, and sequences of the third PCR primer pair are set forth in SEQ ID NOs. 5-6;
2) subjecting the first PCR amplification reaction system, the second PCR amplification reaction system and the third PCR amplification reaction system to amplification respectively under corresponding PCR conditions to obtain a first amplification product, a second amplification product and a third amplification product, and mixing the three amplification products into one tube at a mass ratio of 30:2:3 to obtain a pooled PCR amplification product; and
3) subjecting the pooled PCR amplification product to magnetic bead purification, concentration and fragment size detection, followed by library construction, on-machine library preparation, on-machine sequencing and bioinformatics analysis, whereby the KIR genotype of the test sample can be determined.

6. The method according to claim 5, wherein the PCR amplification reaction systems are respectively as follows:
the first PCR amplification reaction system is 25.0 μL in volume and comprises components of: 2.0 μL of 10 μM the first PCR primer pair, 2.0 μL of 50-100 ng/μL genomic DNA, 12.5 μL of DNA polymerase mixture for rapid amplification of long fragment, and 8.5 μL of ddH₂O;
the second PCR amplification reaction system is 25.0 μL in volume and comprises components of: 2.0 μL of 10 μM the second PCR primer pair, 2.0 μL of 50-100 ng/μL genomic DNA, 12.5 μL of DNA polymerase mixture for rapid amplification of long fragment, and 8.5 μL of ddH₂O; and
the third PCR amplification reaction system is 25.0 μL in volume and comprises components of: 6.0 μL of 1.0 μM the third PCR primer pair, 2.0 μL of 50-100 ng/μL genomic DNA, 12.5 μL of DNA polymerase mixture for rapid amplification of long fragment, and 4.5 μL of ddH₂O.

7. The method according to claim 5, wherein conditions for the PCR amplification are respectively as follows:
reaction program for the first PCR amplification reaction system are: 98° C. for 2 min; 98° C. for 10 Sec; 59° C. for 10 Sec; 72° C. for 4 min, for 35 cycles; and 72° C. for 5 min;
reaction program for the second PCR amplification reaction system are: 98° C. for 2 min; 98° C. for 10 Sec; 56° C. for 10 Sec; 72° C. for 4 min, for 35 cycles; and 72° C. for 5 min; and
reaction program for the third PCR amplification reaction system are: 98° C. for 2 min; 98° C. for 10 Sec; 65° C. for 10 Sec; 72° C. for 5 min, for 35 cycles; and 72° C. for 5 min.

8. The method according to claim 5, wherein lengths of amplification products of the first PCR primer pair, the second PCR primer pair and the third PCR primer pair range from 9.2 kb to 16.7 kb, covering all exons and introns of each KIR gene.
